(19) United States
(12) Patent Application Publication
(10) Pub. No.: US 2022/0071956 A1
(43) Pub. Date: Mar. 10, 2022

(54) COMPOSITION FOR SUPRESSING SECRETION OF EXTRACELLULAR VESICLES

(71) Applicants: NISSAN CHEMICAL CORPORATION, Tokyo (JP); NATIONAL UNIVERSITY CORPORATION KANAZAWA UNIVERSITY, Kanazawa (JP)

(72) Inventors: Kazutaka MATOBA, Shiraoka (JP); Katsuhiko KIDA, Shiraoka (JP); Taito NISHINO, Shiraoka (JP); Rikinari HANAYAMA, Kanazawa (JP); Takeshi YOSHIDA, Kanazawa (JP)

(73) Assignees: NISSAN CHEMICAL CORPORATION, Tokyo (JP); NATIONAL UNIVERSITY CORPORATION KANAZAWA UNIVERSITY, Kanazawa (JP)

(21) Appl. No.: 17/418,686

(22) PCT Filed: Dec. 26, 2019
(86) PCT No.: PCT/JP2019/051079

§ 371 Date: Jun. 25, 2021

(30) Foreign Application Priority Data
Dec. 27, 2018 (JP) 2018-245015

(51) Int. Cl. A61K 31/409; A61P 35/00; A61K 31/404

(52) U.S. Cl. CPC A61K 31/409; A61K 31/404; A61P 35/00

(57) ABSTRACT

The invention provides a composition for suppressing secretion of extracellular vesicle, which comprises a compound having a structure of the formula II (wherein, each substituent is as defined in the specification), or a pharmaceutically acceptable salt thereof, or a compound having a structure of the formula I (wherein, each substituent is as defined in the specification), or a metal complex thereof, or a pharmaceutically acceptable salt thereof.

### TECHNICAL FIELD

The present invention relates to a composition for suppressing secretion of extracellular vesicles.

### BACKGROUND ART

Extracellular vesicles are vesicles secreted by cells, whose membrane structure consists of a lipid bilayer, similar to the cells themselves and organelles in the cells, and it has been known that they are stably present in all body fluids such as saliva, blood, urine, amniotic fluid, etc., and cell culture fluid.

As extracellular vesicles, for example, Exosomes (exosomes), Microvesicles (MV; microvesicles), Apoptotic Bodies (apoptotic bodies), etc., have been known. Exosomes are vesicles of about 20 to about 200 nm derived from endocytosis pathways, and as the constitutional components, proteins, nucleic acids (mRNA, miRNA, non-coding RNA, etc.), etc., have been known, and they can have a function of controlling intercellular communication. Microvesicles (MV) are vesicles of about 50 to about 1,000 nm derived from cytoplasmic membrane, and as the constitutional components, proteins, nucleic acids (mRNA, miRNA, non-coding RNA, etc.), etc., have been known, and they can have a function of controlling intercellular communication. Apoptotic bodies are vesicles of about 500 to about 2,000 nm derived from cytoplasmic membrane, and as the constitutional components, fragmented nuclei, cell organ (organelles), etc., have been known, and they can have a function of inducing phagocytosis, etc.

In recent years, extracellular vesicles have been attracted attention to act as a mediator of intercellular communication in a living body, and to have relationship with diseases such as cancer and neurodegenerative diseases.

In Non-Patent Document 1, it has been reported that exosomes secreted by fibroblasts are involved in a protrusion of lung cancer cells. In Non-Patent Documents 2 and 3, it has been reported that exosomes derived from melanoma promote metastasis of primary lesion, and exosomes, which are secreted from the metastasis by stimulation of neutral sphingomyelinase 2 (nSMase), further promote cancerous metastasis. In Non-Patent Document 4, it has been reported that CD9 or CD63-positive extracellular vesicles are removed by macrophages by administering human specific CD9 or CD63 antibody, whereby metastasis from primary lesion of breast cancer of a mouse model to lung, lymph nodes and thoracic cavity can be suppressed. Also, in Non-Patent Document 5, it has been reported that in ovarian cancer, malignant extracellular vesicles containing MMP1 mRNA induce apoptosis of mesothelial cells of peritoneum, and as a result, it promotes peritoneal metastasis of ovarian cancer. Further, in Non-Patent Document 6, it has been suggested a possibility that development of deposition of tau in Alzheimer's disease can be prevented by suppressing secretion of exosomes from microglia.

From such a background, in recent years, research has been carried out on substances that suppress secretion of extracellular vesicles. For example, in Patent Document 1, it has been reported that a specific compound suppresses an amount of exosomes secreted from melanocytes and exhibits a whitening effect. Also, in Patent Document 2, it has been reported that a substance that suppresses expression of NAPG, HINT3 or GXYLT1 gene, or a substance that inhibits activity of NAPG, HINT3 or GXYLT1 protein inhibits secretion of exosomes.

### PRIOR ART DOCUMENTS

### Patent Documents

Patent Document 1: WO 2017/003114
Patent Document 2: WO 2017/043370

### Non-Patent Documents

Non-Patent Document 1: Luga V, et al, Cell, Dec. 21, 2012, 151(7), 1542-56
Non-Patent Document 2: Peinado H, et al, Nat Med., 2012 June; 18(6): 883-91
Non-Patent Document 3: Nobuyoshi Kosaka, et al, J Biol. Chem, 2013, 288: 10349-10859
Non-Patent Document 4: Nishida-Aoki N, et al, Mol Ther., 2017, 25(1): 181-191
Non-Patent Document 5: Yokoi A, et al, Nat Commun., 2017, 8:14470
Non-Patent Document 6: Asai H, et al, Nat Neurosci., 2015 November; 18(11): 1584-93

### SUMMARY OF THE INVENTION

### Problems to be Solved by the Invention

However, with regard to the substance that suppresses secretion of extracellular vesicles, a number of the reports has been limited, and it has been desired to develop a new composition for suppressing secretion of extracellular vesicles.

Accordingly, an object of the present invention is to provide a composition for suppressing secretion of extracellular vesicles.

### Means to Solve the Problems

In view of the above-mentioned problems, the present inventors have earnestly studied, and as a result, they have found that a composition containing a specific compound unexpectedly suppresses secretion of extracellular vesicles, and completed the present invention.

Accordingly, the present invention provides the following, in summary.

[1] A composition for suppressing secretion of extracellular vesicle, which comprises a compound having a structure of the formula I:

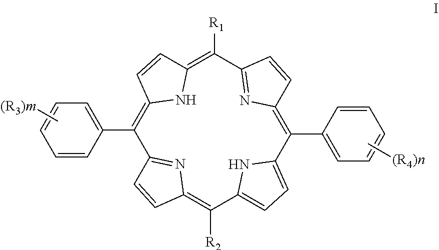

wherein

R₁ and R₂ are independently hydrogen, or phenyl which may be substituted by 1 to 5 substituents selected from the group consisting of halogen, —C(O)R₅, —NR₆R₇, —N⁺R₈R₉R₁₀ and nitro,

each R₃ is independently halogen, —C(O)R₅, —NR₆R₇, —N⁺R₈R₉R₁₀ or nitro,

each R₄ is independently halogen, —C(O)R₅, —NR₆R₇, —N⁺R₈R₉R₁₀ or nitro,

each R₅ is independently hydrogen, hydroxy, or C₁₋₆ alkyl which may be substituted by halogen, hydroxy, amino or nitro,

each R₆, each R₇, each R₈, each R₉ and each R₁₀ are independently hydrogen, or C₁₋₆ alkyl which may be substituted by halogen, hydroxy, amino or nitro,

m is 0 to 5, and

n is 0 to 5,

or a metal complex thereof, or a pharmaceutically acceptable salt thereof, or a compound having a structure of the formula II:

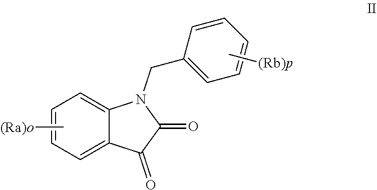

wherein

each Ra is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,

each Rb is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,

o is 0 to 4, and

p is 0 to 5,

or a pharmaceutically acceptable salt thereof.
[2] The composition described in [1], which contains the compound having the structure of the formula I, or a pharmaceutically acceptable salt thereof.
[3] The composition described in [1] or [2], wherein R₁ and R₂ are phenyls which may be substituted by 1 to 5 substituents selected from the group consisting of halogen, —C(O)R₅, —NR₆R₇, —N⁺R₈R₉R₁₀ and nitro.
[4] The composition described in any of [1] to [3], wherein m is 1.
[5] The composition described in [1] or [2], wherein R₁ and R₂ are hydrogen.
[6] The composition described in [1], which contains the compound having the structure of the formula II, or a pharmaceutically acceptable salt thereof.
[7] The composition described in [1] or [6], wherein o is 0.
[8] The composition described in any of [1], [6] and [7], wherein p is 1 or 2.
[9] The composition described in any of [1] and [6] to [8], wherein each Rb is halogen.
[10] The composition described in [1], wherein the compound having the structure of the formula I or the compound having the structure of the formula II is selected from the group consisting of the following:

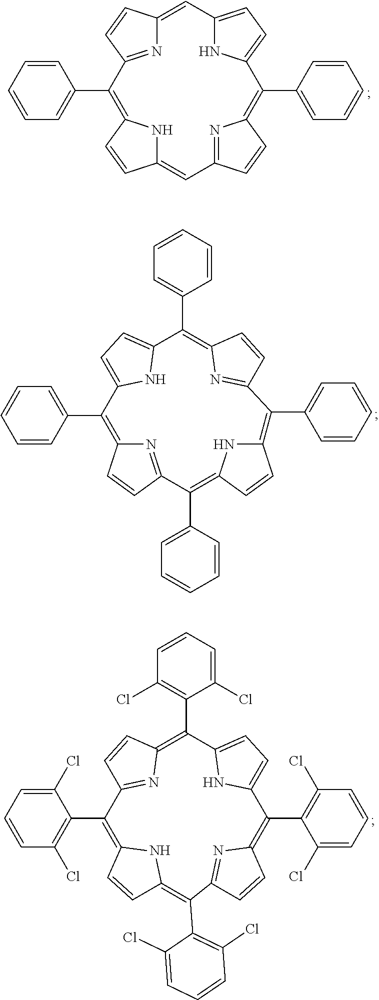

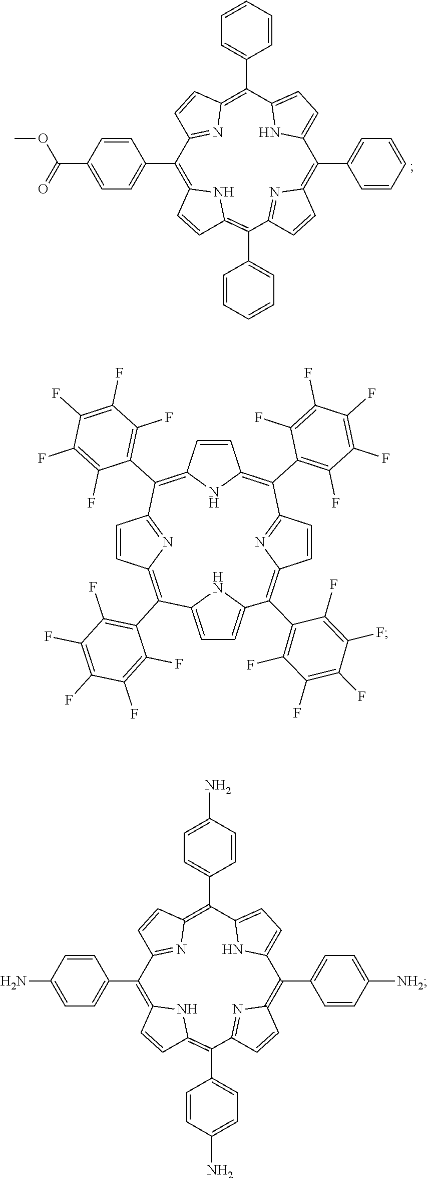 [SMILES: COC(=O)c1ccc(-c2c3nc(c(-c4ccccc4)c4ccc([nH]4)c(-c4ccccc4)c4nc(c(-c5ccccc5)c5ccc2[nH]5)C=C4)C=C3)cc1 ; Fc1c(F)c(F)c(-c2c3nc(c(-c4c(F)c(F)c(F)c(F)c4F)c4ccc([nH]4)c(-c4c(F)c(F)c(F)c(F)c4F)c4nc(c(-c5c(F)c(F)c(F)c(F)c5F)c5ccc2[nH]5)C=C4)C=C3)c(F)c1F ; Nc1ccc(-c2c3nc(c(-c4ccc(N)cc4)c4ccc([nH]4)c(-c4ccc(N)cc4)c4nc(c(-c5ccc(N)cc5)c5ccc2[nH]5)C=C4)C=C3)cc1]

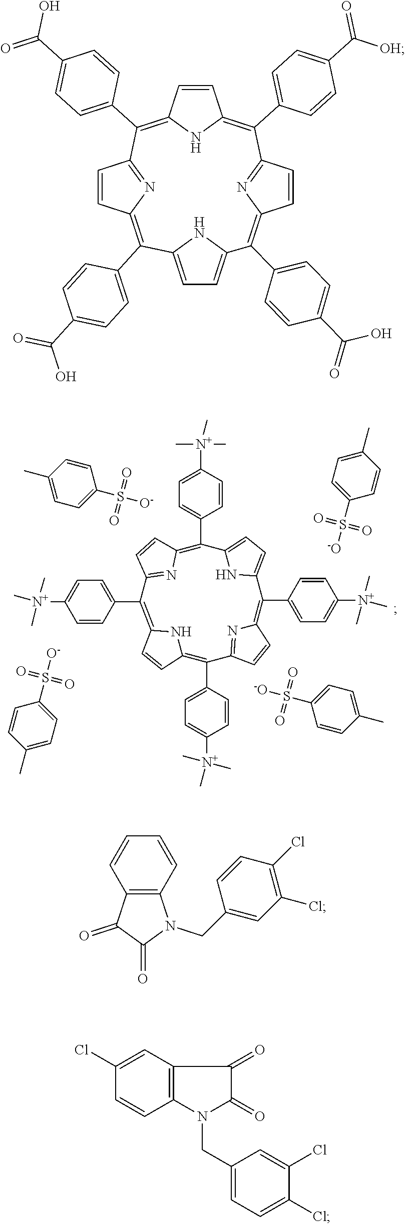

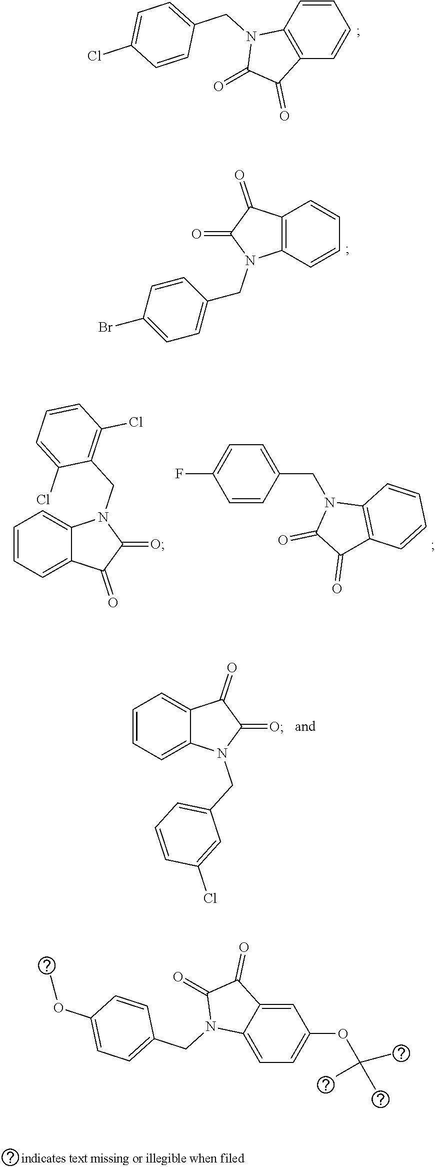 [SMILES: O=C1C(=O)N(Cc2ccc(Cl)cc2)c2ccccc21 ; O=C1C(=O)N(Cc2ccc(Br)cc2)c2ccccc21 ; O=C1C(=O)N(Cc2c(Cl)cccc2Cl)c2ccccc21 ; O=C1C(=O)N(Cc2ccc(F)cc2)c2ccccc21 ; O=C1C(=O)N(Cc2cccc(Cl)c2)c2ccccc21 ; COc1ccc(CN2C(=O)C(=O)c3cc(OC(C)(C)C)ccc32)cc1]

[11] The composition described in any of [1] to [10] for treatment or prophylaxis of cancer.
[12] The composition described in any of [1] to [10] for prophylaxis of cancerous metastasis.
[13] The composition described in any one of [1] to [10] which is a medium composition.
[14] A method for suppressing secretion of extracellular vesicles, which comprises bringing the compound defined in any of [1] to [10], or a metal complex thereof, or a pharmaceutically acceptable salt thereof into contact with cells or tissues in vitro or ex vivo.

### Effects of the Invention

According to the present invention, a composition for suppressing secretion of extracellular vesicle can be provided.

### BRIEF DESCRIPTION OF THE DRAWINGS

FIG. 1A shows the results of evaluating amounts of extracellular vesicles when 0 to 10 μM of the compounds of Examples 1 to 5 or Comparative Examples 2, 3 and 5 were added to U-87MG cells by the Tim4-CD63 ELISA method in Test Example 1-1.

FIG. 1B shows the results of evaluating amounts of extracellular vesicles when 0 to 10 μM of the compounds of Examples 1, 6 to 8 or Comparative Examples 1 and 4 were added to U-87MG cells by the Tim4-CD63 ELISA method in Test Example 1-1.

FIG. 1C shows the results of evaluating amounts of extracellular vesicles when 0 to 10 μM of the compounds of Examples 9 to 16 or Comparative Examples 6 to 8 were added to U-87MG cells by the Tim4-CD9 ELISA method in Test Example 1-1.

FIG. 1D shows the results of evaluating amounts of extracellular vesicles when 0 to 10 μM of the compounds of Examples 9 to 16 or Comparative Examples 6 to 8 were added to U-87MG cells by the Tim4-CD63 ELISA method in Test Example 1-1.

FIG. 1E shows the results of evaluating amounts of extracellular vesicles when 0 to 20 μM of the compound of Example 9 were added to U-87MG cells by the Tim4-CD63 ELISA method in Test Example 1-2.

FIG. 1F shows the results of evaluating amounts of extracellular vesicles when 0 to 10 μM of the compound of Example 9 were added to various cells by the Tim4-ELISA method in Test Example 1-3.

FIG. 2A shows the results when cytotoxicity of 0 to 10 μM of the compounds of Examples 1 to 5 or Comparative Examples 2, 3 and 5, or a dead cell group (sample to which 9 μL of Lysis Buffer was added to compound-untreated cells 15 minutes before recovery of a culture medium) (PC) as a positive control were evaluated by an amount of LDH in the culture supernatant in Test Example 2-1.

FIG. 2B shows the results when cytotoxicity of 0 to 10 μM of the compounds of Examples 1, 6 to 8 or Comparative Examples 1 and 4, or a dead cell group (sample to which 9 μL of Lysis Buffer was added to compound-untreated cells 15 minutes before recovery of a culture medium) (PC) as a positive control were evaluated by an amount of LDH in the culture supernatant in Test Example 2-1.

FIG. 2C shows the results when cytotoxicity of 0 to 10 μM of the compounds of Examples 9 to 16 or Comparative Examples 6 to 8, or a dead cell group (sample to which 9 μL of Lysis Buffer was added to compound-untreated cells 15 minutes before recovery of a culture medium) (PC) as a positive control were evaluated by an amount of LDH in the culture supernatant, and shown as % of the absorbance relative to PC in Test Example 2-1.

FIG. 2D shows the results when cytotoxicity of 0 to 40 μM of the compound of Example 9 or a dead cell group (sample to which 9 μL of Lysis Buffer was added to compound-untreated cells 15 minutes before recovery of a culture medium) (PC) as a positive control were evaluated by an amount of LDH in the culture supernatant in Test Example 2-1.

FIG. 2E shows the results of cytotoxicity of 10 μM of the compound of Example 9 in various cells and shown as % of cytotoxicity of solvent alone (0 μM) in Test Example 2-2.

FIG. 3A shows the results of evaluating amounts of extracellular vesicles when 0 to 20 μM of the compound of Example 9 were added to U-87MG cells by Nano SIGHT in Test Example 3.

FIG. 3B shows the results of evaluating amounts of extracellular vesicles when 0 or 10 μM of the compounds of Example 9 and 11 to 15 were added to U-87MG cells by Nano SIGHT in Test Example 3.

FIG. 4 shows the results of evaluating amounts of extracellular vesicles when 0 to 20 μM of the compound of Example 9 were added to U-87MG cells by the Tim4-CD63 ELISA method in Test Example 4.

### EMBODIMENTS TO CARRY OUT THE INVENTION

The present invention is to provide a composition for suppressing secretion of extracellular vesicle, which comprises a compound having a structure of the formula I:

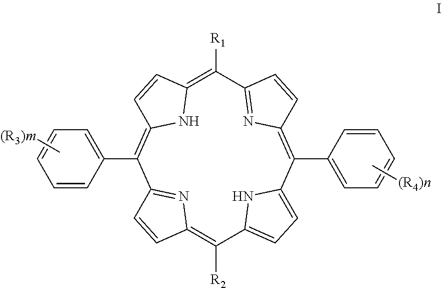

wherein

R₁ and R₂ are independently hydrogen, or phenyl which may be substituted by 1 to 5 substituents selected from the group consisting of halogen, —C(O)R₅, —NR₆R₇, —N⁺R₈R₉R₁₀ and nitro,

each R₃ is independently halogen, —C(O)R₅, —NR₆R₇, —N⁺R₈R₉R₁₀ or nitro,

each R₄ is independently halogen, —C(O)R₅, —NR₆R₇, —N⁺R₈R₉R₁₀ or nitro,

each R₅ is independently hydrogen, hydroxy, or C₁₋₆ alkyl which may be substituted by halogen, hydroxy, amino or nitro,

each R₆, each R₇, each R₈, each R₉ and each R₁₀ are independently hydrogen, or C₁₋₆ alkyl which may be substituted by halogen, hydroxy, amino or nitro,

m is 0 to 5, and

n is 0 to 5,

or a metal complex thereof, or a pharmaceutically acceptable salt thereof, or a compound having a structure of the formula II:

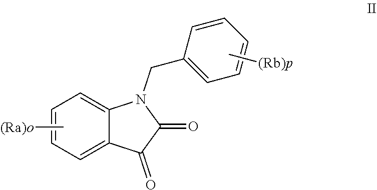

wherein

each Ra is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,

each Rb is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,

o is 0 to 4, and

p is 0 to 5,

or a pharmaceutically acceptable salt thereof.

The present invention is also to provide a compound having the structure of the formula I, or a metal complex thereof, or a pharmaceutically acceptable salt thereof, or a compound having the structure of the formula II, or a pharmaceutically acceptable salt thereof for suppressing secretion of extracellular vesicles.

The present invention is also to provide use of a compound having the structure of the formula I, or a metal complex thereof, or a pharmaceutically acceptable salt thereof, or a compound having the structure of the formula II, or a pharmaceutically acceptable salt thereof, in the manufacture of a composition for suppressing secretion of extracellular vesicle.

The present invention is also to provide a method for suppressing secretion of extracellular vesicles, which comprises administering a compound having the structure of the formula I, or a metal complex thereof, or a pharmaceutically acceptable salt thereof, or a compound having the structure of the formula II, or a pharmaceutically acceptable salt thereof to a subject who requires it.

The term “halogen” as used in the present specification means, but is not limited thereto, for example, fluorine, chlorine, bromine, iodine and the like.

The term “nitro” as used in the present specification means a group represented by the formula “—NO₂”.

The terms “phenyl which may be substituted by 1 to 5 substituents selected from the group consisting of halogen, —C(O)R₅, —NR₆R₇, —N⁺R₈R₉R₁₀ and nitro” as used in the present specification mean a group in which 1 to 5 hydrogen atoms of phenyl may be or may not be substituted by —C(O)R₅, —NR₆R₇, —N⁺R₈R₉R₁₀ or nitro (in these formulae, R₆ to R₁₀ are as defined in the present specification).

The term “hydroxy” as used in the present specification means a group represented by the formula “—OH”.

The term “amino” as used in the present specification means a group represented by the formula “—NH₂”.

The terms “C₁₋₆ alkyl” as used in the present specification mean a saturated linear or branched hydrocarbon group having 1 to 6 carbon atoms (which are not limited to these, and it contains, for example, methyl, ethyl, propyl, isopropyl, n-butyl, i-butyl, t-butyl, pentyl, hexyl, etc.). Preferable C₁₋₆ alkyl is C₁₋₄ alkyl (for example, it contains methyl, ethyl, propyl, butyl, isopropyl, etc.), and more preferably methyl.

The terms “C₁₋₆ alkyl which may be substituted by halogen, hydroxy, amino or nitro” as used in the present specification mean a group in which one or more hydrogen atoms of C₁-C₆ alkyl may be substituted by halogen, hydroxy, amino or nitro, or may not be substituted.

The terms “C₁₋₆ alkoxy” as used in the present specification mean a group represented by the formula “—O—C₁₋₆ alkyl” (which are not limited to these, and it contains, for example, methoxy, ethoxy, propoxy, isopropoxy, etc.). Preferable C₁₋₆ alkoxy is methoxy, ethoxy or propoxy, and more preferably methoxy.

The terms “C₁₋₆ alkoxy which may be substituted by halogen, hydroxy, amino or nitro” as used in the present specification mean a group in which one or more hydrogen atoms in C₁₋₆ alkoxy may be substituted by halogen, hydroxy, amino or nitro, or may not be substituted.

The terms “metal complex” as used in the present specification mean a metal complex in which a central metal is coordinated to a porphine ring in the compound having the structure of the formula I. Examples of the central metal capable of forming a metal complex may be mentioned, which is not limited to these, there may be mentioned, for example, iron, zinc, copper, gold, cobalt, nickel, chromium, magnesium, etc.

The terms “pharmaceutically acceptable salt” as used in the present specification mean, which is not limited thereto, there may be mentioned, for example, acid addition salts with inorganic acids (which are not limited to these, there may be mentioned, for example, hydrochloric acid, hydrobromic acid, sulfuric acid, nitric acid, carbonic acid, phosphoric acid, etc.) or organic acids (which are not limited to these, there may be mentioned, for example, formic acid, acetic acid, propionic acid, glycolic acid, gluconic acid, lactic acid, mesylic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicylic acid, etc.); salts with metals (which are not limited to these, there may be mentioned, for example, sodium, potassium, calcium, magnesium, iron, zinc, copper, manganese); ammonium salts; salts with organic bases (which are not limited to these, there may be mentioned, for example, isopropylamine, trimethylamine, diethylamine, triethylamine, tripropylamine, ethanolamine, 2-diethylaminoethanol, trimetamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, etc.), and the like.

A compound having the structure of the formula I, or a metal complex thereof, or a pharmaceutically acceptable salt thereof, or a compound having the structure of the formula II, or a pharmaceutically acceptable salt thereof (contained in the composition described in the present specification) include a solvate (for example, a hydrate thereof), polymorphs thereof, etc.

The term “solvate” as used in the present specification means an associated body, a complex, etc., of one or more solvent molecules and a compound having the structure of the formula I, or a metal complex thereof, or a pharmaceutically acceptable salt thereof, or a compound having the structure of the formula II, or a pharmaceutically acceptable salt thereof. As the solvent, which is not limited thereto, there may be mentioned, for example, water, methanol, ethanol, isopropanol, DMSO, acetic acid, ethyl acetate, etc.

In one embodiment of the present invention, provided is a composition described in the present specification which comprises the compound having the structure of the formula I, or a metal complex thereof, or a pharmaceutically acceptable salt thereof.

In another embodiment of the present invention, provided is a compound having the structure of the formula I, or a metal complex thereof, or a pharmaceutically acceptable salt thereof for suppressing secretion of extracellular vesicles.

In another embodiment of the present invention, provided is use of a compound having the structure of the formula I, or a metal complex thereof, or a pharmaceutically acceptable salt thereof, in the manufacture of a composition for suppressing secretion of extracellular vesicle.

In another embodiment of the present invention, provided is a method for suppressing secretion of extracellular vesicles, which comprises administering a compound having the structure of the formula I, or a metal complex thereof, or a pharmaceutically acceptable salt thereof to a subject who requires it.

In one embodiment of the present invention, provided is a composition (or a compound, use or method) described in the present specification wherein R₁ is phenyl which may be substituted by 1 to 5 substituents selected from the group consisting of halogen, —C(O)R₅, —NR₆R₇, —N⁺R₈R₉R₁₀ and nitro.

In one embodiment of the present invention, provided is a composition (or a compound, use or method) described in the present specification wherein R₁ is phenyl which may be substituted by 1 to 5 —N⁺R₈R₉R₁₀.

In one embodiment of the present invention, provided is a composition (or a compound, use or method) described in the present specification wherein R₁ is phenyl substituted by one —N⁺R₈R₉R₁₀.

In one embodiment of the present invention, provided is a composition (or a compound, use or method) described in the present specification wherein R₂ is phenyl which may be substituted by 1 to 5 substituents selected from the group consisting of halogen, —C(O)R₅, —NR₆R₇, —N⁺R₈R₉R₁₀ and nitro.

In one embodiment of the present invention, provided is a composition (or a compound, use or method) described in the present specification wherein R₂ is phenyl which may be substituted by 1 to 5 —N⁺R₈R₉R₁₀.

In one embodiment of the present invention, provided is a composition (or a compound, use or method) described in the present specification wherein R₂ is phenyl substituted by one —N⁺R₈R₉R₁₀.

In preferable one embodiment of the present invention, provided is a composition (or a compound, use or method) described in the present specification wherein R₁ and R₂ are phenyl which may be substituted by 1 to 5 substituents selected from the group consisting of halogen, —C(O)R₅, —NR₆R₇, —N⁺R₈R₉R₁₀ and nitro.

In preferable one embodiment of the present invention, provided is a composition (or a compound, use or method) described in the present specification wherein R₁ and R₂ are phenyls which may be substituted by 1 to 5 —N⁺R₈R₉R₁₀.

In preferable one embodiment of the present invention, provided is a composition (or a compound, use or method) described in the present specification wherein R₁ and R₂ are phenyls substituted by one —N⁺R₈R₉R₁₀.

In one embodiment of the present invention, provided is a composition (or a compound, use or method) described in the present specification wherein m is 1 or 2.

In one embodiment of the present invention, provided is a composition (or a compound, use or method) described in the present specification wherein m is 1.

In one embodiment of the present invention, provided is a composition (or a compound, use or method) described in the present specification wherein n is 1 or 2.

In one embodiment of the present invention, provided is a composition (or a compound, use or method) described in the present specification wherein n is 1.

In one embodiment of the present invention, provided is a composition (or a compound, use or method) described in the present specification wherein R₁ is hydrogen.

In one embodiment of the present invention, provided is a composition (or a compound, use or method) described in the present specification wherein R₂ is hydrogen.

In preferable one embodiment of the present invention, provided is a composition (or a compound, use or method) described in the present specification R₁ and R₂ are hydrogen.

In one embodiment of the present invention, provided is a composition described in the present specification which contains a compound having the structure of the formula II, or a pharmaceutically acceptable salt thereof.

In another embodiment of the present invention, provided is a compound having the structure of the formula II, or a pharmaceutically acceptable salt thereof for suppressing secretion of extracellular vesicles.

In another embodiment of the present invention, provided is use of a compound having the structure of the formula II, or a pharmaceutically acceptable salt thereof, in the manufacture of a composition for suppressing secretion of extracellular vesicle.

In another embodiment of the present invention, provided is a method for suppressing secretion of extracellular vesicles, which comprises administering a compound having the structure of the formula II, or a pharmaceutically acceptable salt thereof to a subject who requires it.

In one embodiment of the present invention, provided is a composition (or a compound, use or method) described in the present specification wherein o is 0.

In one embodiment of the present invention, provided is a composition (or a compound, use or method) described in the present specification wherein p is 1 or 2.

In one embodiment of the present invention, provided is a composition (or a compound, use or method) described in the present specification wherein p is 2.

In one embodiment of the present invention, provided is a composition (or a compound, use or method) described in the present specification wherein each Rb is halogen.

In one embodiment of the present invention, provided is a composition (or a compound, use or method) described in the present specification wherein each Rb is chlorine.

In preferable one embodiment of the present invention, provided is a composition (or a compound, use or method) described in the present specification wherein p is 2, and each Rb is halogen.

In preferable one embodiment of the present invention, provided is a composition (or a compound, use or method) described in the present specification wherein p is 2, and each Rb is chlorine.

In one embodiment of the present invention, provided is a composition (or a compound, use or method) described in the present specification wherein the compound having the structure of the formula I or the compound having the structure of the formula II is selected from the group consisting of the following:

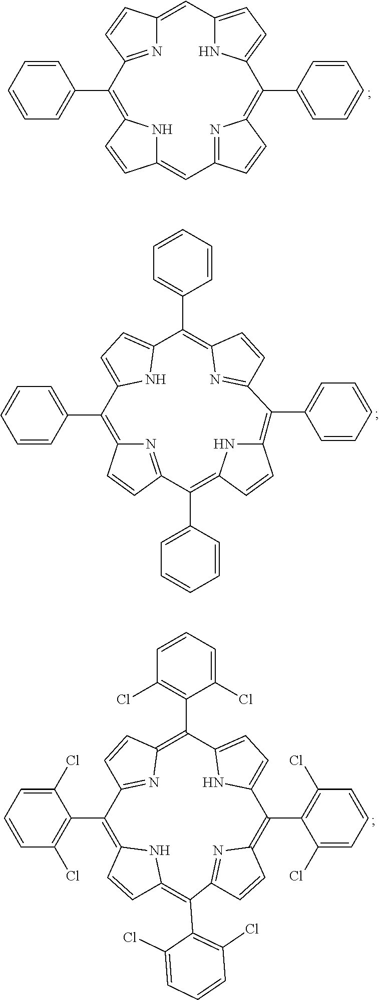

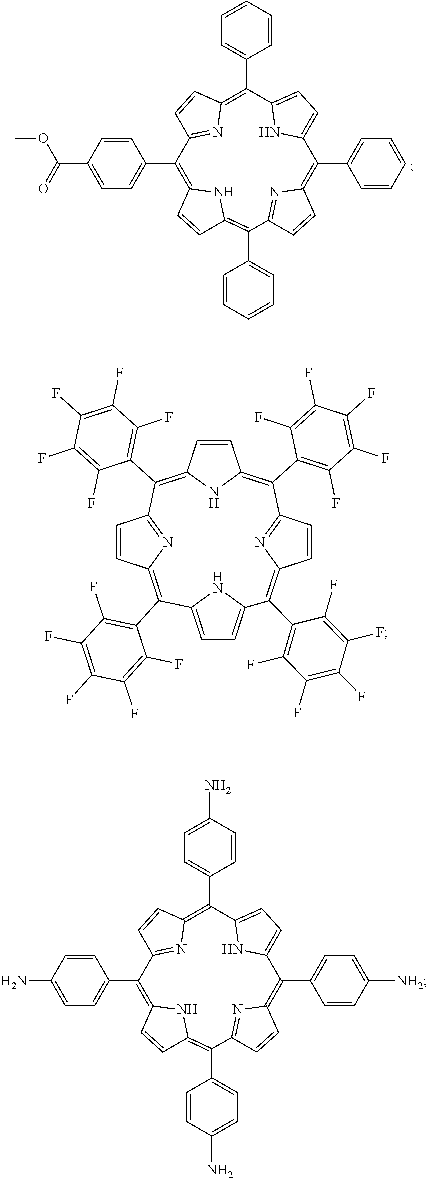 [SMILES: COC(=O)c1ccc(-c2c3nc(c(-c4ccccc4)c4ccc([nH]4)c(-c4ccccc4)c4nc(c(-c5ccccc5)c5ccc2[nH]5)C=C4)C=C3)cc1 ; Fc1c(F)c(F)c(-c2c3nc(c(-c4c(F)c(F)c(F)c(F)c4F)c4ccc([nH]4)c(-c4c(F)c(F)c(F)c(F)c4F)c4nc(c(-c5c(F)c(F)c(F)c(F)c5F)c5ccc2[nH]5)C=C4)C=C3)c(F)c1F ; Nc1ccc(-c2c3nc(c(-c4ccc(N)cc4)c4ccc([nH]4)c(-c4ccc(N)cc4)c4nc(c(-c5ccc(N)cc5)c5ccc2[nH]5)C=C4)C=C3)cc1]

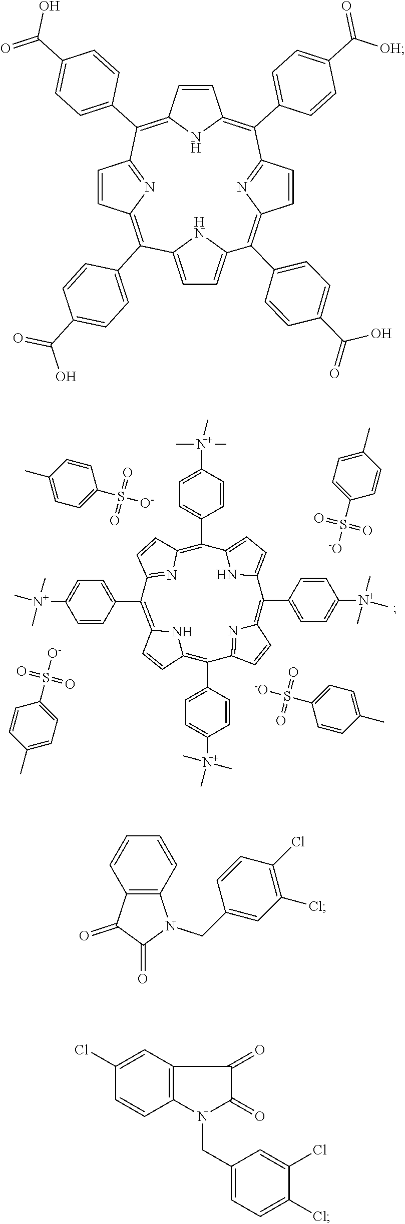

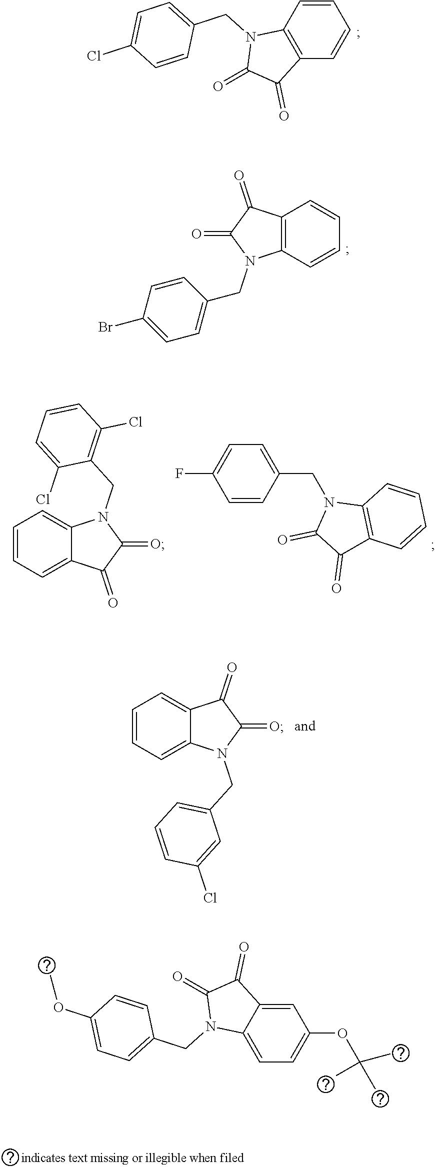 [SMILES: O=C1C(=O)N(Cc2ccc(Cl)cc2)c2ccccc21 ; O=C1C(=O)N(Cc2ccc(Br)cc2)c2ccccc21 ; O=C1C(=O)N(Cc2c(Cl)cccc2Cl)c2ccccc21 ; O=C1C(=O)N(Cc2ccc(F)cc2)c2ccccc21 ; O=C1C(=O)N(Cc2cccc(Cl)c2)c2ccccc21 ; COc1ccc(CN2C(=O)C(=O)c3cc(OC(C)(C)C)ccc32)cc1]

The compound having the structure of the formula I, or a metal complex thereof, or a pharmaceutically acceptable salt thereof, or the compound having the structure of the formula II, or a pharmaceutically acceptable salt thereof are commercially available, or can be produced by a well-known or universally known method or a method similar thereto. For example, these compounds can be purchased from Pharmeks Ltd., Enamine Ltd., etc.

The terms “extracellular vesicles” used in the present specification are not specifically limited as long as they are vesicles secreted from cells, and there may be mentioned, for example, Exosomes (exosomes), Microvesicles (MV; microvesicles), Apoptotic Bodies (apoptotic bodies), etc.

The term “exosomes” used in the present specification means vesicles of about 20 to about 200 nm derived from endocytosis pathways. As the constitutional components of exosomes, there may be mentioned, for example, proteins, nucleic acids (mRNA, miRNA, non-coding RNA), etc. Exosomes can have a function of controlling intercellular communication. Examples of the marker molecule of exosomes may be mentioned, for example, Alix, Tsg101, tetraspanin (for example, CD81, CD63, CD9), flotillin, phosphatidylserine, etc.

The term “microvesicles” used in the present specification means vesicles of about 50 to about 1,000 nm derived from cytoplasmic membrane. As the constitutional components of microvesicles, there may be mentioned, for example, proteins, nucleic acids (mRNA, miRNA, non-coding RNA, etc.), etc. Microvesicles can have a function of controlling intercellular communication, etc. Examples of the marker molecule of microvesicles may be mentioned, for example, integrin, selectin, CD40, CD154, etc.

The terms “apoptotic body” used in the present specification mean vesicles of about 500 to about 2,000 nm derived from cytoplasmic membrane. As the constitutional components of apoptotic body, there may be mentioned, for example, fragmented nuclei, cell organ (organelles), etc. Apoptotic body can have a function of inducing phagocytosis, etc. Examples of the marker molecule of apoptotic body may be mentioned, for example, Annexin V, phosphatidylserine, etc.

In one embodiment of the present invention, provided is a composition (or a compound, use or method) described in the present specification wherein the extracellular vesicles are exosomes.

In one embodiment of the present invention, provided is a composition (or a compound, use or method) described in the present specification wherein the extracellular vesicles are CD9, CD63 and/or CD81 positive extracellular vesicles (preferably CD9, CD63 and/or CD81 positive exosomes).

The cells that secrete extracellular vesicles are not particularly limited as long as they can secrete extracellular vesicles, and there may be mentioned, for example, animal-derived cells including skin cells such as epidermal cells (keratinocytes, etc.), pigment cells (melanocytes, etc.), basal cells, prickle cells, granule cells, corneocytes, fibroblasts, mast cells, etc.; brain cells such as neural stem cells, neuroglial cells, nerve cells, microglia, etc.; cells derived from adipose tissues such as adipose cells (including white adipose cells, brown adipose cells, etc.), mesenchymal stem cell, etc.; lymphocytes (T cells, B cells, NK cells, etc.), monocytes, epithelial cells, endothelial cells, muscle cells, nerve cells, fibroblasts, hair cells, hepatocytes, gastric mucosal cells, intestinal cells, splenic cells, pancreatic cells (pancreatic exocrine cells, etc.), pneumocytes, nephrocytes, mesenchymal cells, etc., other than the above; tissue precursor cells; hematopoietic stem cells, mesenchymal stem cell (including those derived from bone marrow, those derived from placental tissue, those derived from umbilical cord tissue, those derived from dental pulp, etc.), other tissue stem cells (somatic stem cells); etc., and plant-derived cells such as soft tissue cells, collenchyma tissue cells, sclerenchyma tissue cells, xylem cells, phloem cells, epidermal cells, etc., and these may be cells in organisms, primary cultured cells, subcultured cells or established cells, and these may be normal cells, diseased cells including cancerous or tumorigenic cells. The cells that secrete extracellular vesicles are preferably animal-derived cells, more preferably skin cells, or diseased cells including cancerous or tumorigenic cells, and further preferably cancerous or tumorigenic cells.

In one embodiment of the present invention, provided is a composition (or a compound, use or method) described in the present specification for suppressing secretion of extracellular vesicles from animal-derived cells.

In one embodiment of the present invention, provided is a composition (or a compound, use or method) described in the present specification for suppressing secretion of extracellular vesicles from skin cells, or diseased cells including cancerous or tumorigenic cells.

In one embodiment of the present invention, provided is a composition (or a compound, use or method) described in the present specification for suppressing secretion of extracellular vesicles from cancerous or tumorigenic cells.

The composition described in the present specification can suppress secretion of extracellular vesicles, so that it can be used for treatment or prophylaxis of diseases in which extracellular vesicles may be involved.

Diseases that may involve in extracellular vesicles may be mentioned, for example, cancer, neurodegenerative diseases, etc., whereas it is not limited to these.

In one embodiment of the present invention, provided is a composition (or a compound, use or method) described in the present specification for treatment or prophylaxis of cancer or neurodegenerative diseases.

In one embodiment of the present invention, provided is a composition (or a compound, use or method) described in the present specification for treatment or prophylaxis of cancer.

In one embodiment of the present invention, provided is a composition (or a compound, use or method) described in the present specification for treatment or prophylaxis of neurodegenerative diseases.

It has been reported that extracellular vesicles may be involved in cancerous metastasis, exacerbation, etc. Accordingly, in one embodiment of the present invention, provided is a composition (or a compound, use or method) described in the present specification for prophylaxis of cancerous metastasis.

As the cancer, any of the solid cancers and blood cancers are contained, which are not limited to these, and there may be mentioned, for example, small cell lung cancer, non-small cell lung cancer, breast cancer, esophageal cancer, stomach cancer, small intestine cancer, large intestine cancer, colon cancer, rectal cancer, pancreatic cancer, prostatic cancer, bone marrow cancer, kidney cancer (including nephrocyte cancer, etc.), parathyroid cancer, adrenal cancer, ureter cancer, liver cancer, bile duct cancer, uterine cervix cancer, ovarian cancer (for example, its tissue type is serous adenocarcinoma, mucous adenocarcinoma, clear cell adenocarcinoma, etc.), testicular cancer, bladder cancer, vulvar cancer, penile cancer, thyroid cancer, head and neck cancer, craniopharyngeal cancer, pharyngeal cancer, tongue cancer, skin cancer, Merkel cell cancer, melanoma (malignant melanoma, etc.), epithelial cancer, squamous epithelial cell cancer, basal cell cancer, childhood cancer, unknown primary cancer, fibrosarcoma, mucosal sarcoma, liposarcoma, chondrosarcoma, osteogenic sarcoma, chordoma, angiosarcoma, lymphangiosarcoma, lymphangioendothelioma, Kaposi's sarcoma, leiomyosarcoma, rhabdomyosarcoma, synovioma, mesothelioma, Ewing tumor, seminoma, Wilms tumor, brain tumor, glioma, glioblastoma, astrocytoma, myeloblastoma, meningioma, neuroblastoma, medulloblastoma, retinoblastoma, spinal neoplasm, malignant lymphoma (for example, non-Hodgkin lymphoma, Hodgkin lymphoma, etc.), monocytic leukemia (chronic or acute), chronic or acute lymphocytic leukemia, adult T cell leukemia, etc.

As the neurodegenerative diseases, which are not limited to these, there may be mentioned, for example, Alzheimer's disease, Parkinson's disease, cerebral atrophic lateral sclerosis, spinocerebellar degeneration, Frontotemporal lobar degeneration, progressive supranuclear palsy, corticobasal degeneration, Huntington's disease, dystonia, prion disease, multiple-system atrophy, Lewy body disease, polyglutamine disease, etc.

As the subject to be tested which became a subject to which secretion of extracellular vesicles is suppressed, which are not limited to these, there may be mentioned, for example, animals such as mammals, etc., including rodents such as mice, rats, hamsters, guinea pigs, etc.; Lagomorpha such as rabbits, etc.; ungulates such as pigs, cows, goats, horses, sheep, etc.; Carnivora such as dogs, cats, etc.; primates such as humans, monkeys, rhesus monkey, crab-eating monkey, marmoset, orangutan, chimpanzee, etc.; and plants, preferably animals, more preferably rodents or primates, further preferably primates, and more further preferably humans.

In one embodiment of the present invention, the composition described in the present specification is a pharmaceutical, edible or cosmetic composition.

When the composition described in the present specification is used, for example, as a pharmaceutical, edible or cosmetic composition, whereas it is not limited to these, for example, it may contain additives such as excipients, lubricants, binders, disintegrants, pH regulators, solvents, solubilizing aids, suspending agents, tonicity agents, buffers, analgesics, preservatives, antioxidants, colorants, sweeteners, surfactants, etc.

As the excipients, which are not limited to these, there may be mentioned, for example, lactose hydrate, white sugar, glucose, starch, sucrose, crystalline cellulose, mannitol, etc., and these may be used alone or may be used in combination of two or more kinds.

As the lubricants, which are not limited to these, there may be mentioned, for example, light anhydrous silicic acid, stearic acid, magnesium stearate, calcium stearate, sucrose fatty acid ester, polyethylene glycol, talc, etc., and these may be used alone or may be used in combination of two or more kinds.

As the binders, which are not limited to these, there may be mentioned, for example, gum arabic, crystalline cellulose, white sugar, mannitol, dextrin, hydroxypropyl cellulose, hydroxymethyl cellulose, polyvinylpyrrolidone, etc., and these may be used alone or may be used in combination of two or more kinds.

As the disintegrator, which are not limited to these, there may be mentioned, for example, starch, carboxymethyl cellulose, carboxymethyl cellulose calcium, croscarmellose sodium, croscarmellose calcium, carboxymethyl starch sodium, crospovidone, low-substitution degree hydroxypropyl cellulose, etc., and these may be used alone or may be used in combination of two or more kinds.

As the pH regulators, which are not limited to these, there may be mentioned, for example, acetic acid, lactic acid, tartaric acid, oxalic acid, glycolic acid, malic acid, citric acid, succinic acid, fumaric acid, phosphoric acid, hydrochloric acid, sulfuric acid, nitric acid and a salt thereof, sodium hydroxide, potassium hydroxide, sodium hydrogen carbonate, potassium carbonate, etc., and these may be used alone or may be used in combination of two or more kinds.

As the solvents, which are not limited to these, there may be mentioned, for example, water such as tap water, normal water, distilled water, purified water, water for injection, etc.; alcohols such as methanol, ethanol, propanol, isopropanol, etc.; acetone; single fatty acids such as acetic acid, propanoic acid, butanoic acid, pentanoic acid, hexanoic acid, heptanoic acid, myristic acid, stearic acid, oleic acid, etc., or an ester thereof; vegetable oils such as sesame oil, peanut oil, coconut oil, palm oil, soybean oil, olive oil, coconut butter, corn oil, cottonseed oil, castor oil, rapeseed oil, sunflower oil, etc.; propylene glycol; macrogol, etc., and these may be used alone or may be used in combination of two or more kinds.

As the solubilizing aids, which are not limited to these, there may be mentioned, for example, polyethylene glycol; propylene glycol; cyclodextrin; sugar alcohols such as mannitol, etc.; benzyl benzoate; trisaminomethane; cholesterol; triethanolamine; sodium carbonate; sodium citrate; alcohols such as methanol, ethanol, propanol, isopropanol, etc.; single fatty acids such as acetic acid, propanoic acid, butanoic acid, pentanoic acid, hexanoic acid, heptanoic acid, myristic acid, stearic acid, oleic acid, etc., or an ester thereof; vegetable oils such as sesame oil, peanut oil, coconut oil, palm oil, soybean oil, olive oil, coconut butter, corn oil, cottonseed oil, castor oil, rapeseed oil, sunflower oil, etc., and these may be used alone or may be used in combination of two or more kinds.

As the suspending agents, which are not limited to these, there may be mentioned, for example, stearyltriethanolamine, sodium lauryl sulfate, laurylamino-propionic acid, lecithin, glycerin monostearate, polyvinyl alcohol, polyvinylpyrrolidone, carboxymethylcellulose sodium, methylcellulose, hydroxymethyl cellulose, hydroxyethyl cellulose, hydroxypropyl cellulose, etc., and these may be used alone or may be used in combination of two or more kinds.

As the tonicity agents, which are not limited to these, there may be mentioned, for example, sodium chloride, glycerin, mannitol, etc., and these may be used alone or may be used in combination of two or more kinds.

As the buffers, which are not limited to these, there may be mentioned, for example, buffer solutions such as phosphates, acetates, carbonates, citrates, etc., and these may be used alone or may be used in combination of two or more kinds.

As the analgesics, which are not limited to these, there may be mentioned, for example, benzyl alcohol, etc.

As the preservatives, which are not limited to these, there may be mentioned, for example, sorbic acid, potassium sorbate, calcium sorbate, benzoic acid, sodium benzoate, propionic acid, sodium propionate, calcium propionate, sodium dehydroacetate, natamycin, pimaricin, polylysine, nisin, isopropyl paraoxybenzoate, isopropyl parahydroxybenzoate, isopropylparaben, etc., and these may be used alone or may be used in combination of two or more kinds.

As the antioxidants, which are not limited to these, there may be mentioned, for example, sulfites, ascorbic acid, etc., and these may be used alone or may be used in combination of two or more kinds.

As the colorants, which are not limited to these, there may be mentioned, for example, yellow ferric oxide, black iron oxide, food yellow No. 4, food red No. 3, tar pigment, caramel, titanium oxide, riboflavins, etc., and these may be used alone or may be used in combination of two or more kinds.

As the sweeteners, which are not limited to these, there may be mentioned, for example, sugars such as sucrose, fructose, etc.; sugar alcohols such as xylitol, sorbitol, etc.; artificial sweeteners such as aspartame, acesulfame potassium, sucralose, etc., and these may be used alone or may be used in combination of two or more kinds.

As the surfactants, which are not limited to these, there may be mentioned, for example, polysorbates, sodium lauryl sulfate, polyoxyethylene hydrogenated castor oil, etc., and these may be used alone or may be used in combination of two or more kinds.

The composition described in the present specification can be formulated with the above-mentioned additives by the methods known per se, for example, to formulations such as tablets, coated tablets, orally disintegrating tablets, chewable agents, pills, granules, fine granules, powders, hard capsules, soft capsules, liquids (for example, including syrups, injections, lotions, etc.), suspensions, emulsions, jelly agents, patches, ointments, creams, inhalants, suppositories, etc. These may be oral preparations or parenteral preparations. The formulated material may contain, not only the compound having the structure of the formula I, or a metal complex thereof, or a pharmaceutically acceptable salt thereof, or the compound having the structure of the formula II, or a pharmaceutically acceptable salt thereof, but also beneficial other components (for example, therapeutically beneficial other components), depending on the purpose.

In the case of the tablets, for example, it can be formulated as follows.

The compound having the structure of the formula I, or a metal complex thereof, or a pharmaceutically acceptable salt thereof, or the compound having the structure of the formula II, or a pharmaceutically acceptable salt thereof, and an excipient, a disintegrator, a binder, etc., are mixed and granulated with water. The obtained granules are dried and the granules are pulverized depending on necessity. Then, a lubricant, etc., is added thereto and the mixture is further mixed, and this is subjected to compression molding to obtain a tablet.

In the case of the hard capsules, for example, it can be formulated as follows.

The compound having the structure of the formula I, or a metal complex thereof, or a pharmaceutically acceptable salt thereof, or the compound having the structure of the formula II, or a pharmaceutically acceptable salt thereof, and an excipient, etc., are mixed, a lubricant, etc., is added thereto, and the mixture is further mixed. Then, the obtained mixture is filled in a hard capsule (for example, a gelatin capsule, etc.) to obtain a hard capsule.

A content of the compound having the structure of the formula I, or a metal complex thereof, or a pharmaceutically acceptable salt thereof, or the compound having the structure of the formula II, or a pharmaceutically acceptable salt thereof in the composition described in the present specification or in the formulation thereof can be, for example, about 0.01 to about 99.9% by mass, preferably about 0.1 to about 80% by mass, more preferably about 1 to about 50% by mass based on the entire composition or formulation thereof.

An administration dose of the composition described in the present specification or a formulation thereof can be appropriately determined in consideration of sex, age, body weight, health condition, degree of medical condition or diet of the subject to be administered; administration time; administration method; combination of the other drugs; and other factors.

An administration dose of the composition described in the present specification or a formulation thereof is not specifically limited and, for example, as the compound having the structure of the formula I, or a metal complex thereof, or a pharmaceutically acceptable salt thereof, or the compound having the structure of the formula II, or a pharmaceutically acceptable salt thereof, it can be about 0.01 to about 10 mg/kg body weight, preferably about 0.05 to about 5 mg/kg body weight, more preferably about 0.1 to about 1 mg/kg body weight, per a day. These may be administered in a single dose, or may be administered by dividing into two or more times. Provided that, depending on necessity, it may be exceeded the range of the above-mentioned administration dose.

The administration schedule can be determined in consideration of the sex, age, body weight, health condition, degree of medical condition or diet of the subject to be administered; administration time; administration method; combination with other medicines; and other factors, and may be mentioned, for example, every day, once every two days, once every three days, once a week, once a month, once every three months, once every six months, etc.

The formulated material as mentioned above may be used, for example, as an anticancer agent or an anti-neurodegenerative disease agent.

The composition described in the present specification or a formulation thereof exerts a synergistic effect, for example, when used in combination with the other anticancer agents, etc., and in some cases, it can be expected to obtain improvement in therapeutic effect and therapeutic result, shortening of treatment period, reduction of concentration of other anticancer drugs, suppression of side effects, cost reduction, etc.

As the composition described in the present specification or the other anticancer agents that can be used in combination with formulations thereof, which are not limited to these, there may be mentioned, for example, tyrosine kinase inhibitors such as imatinib, nilotinib, dasatinib, gefitinib, erlotinib, lapatinib, sorafenib, sunitinib, crizotinib, axitinib, etc.; antibodies such as trastuzumab, cetuximab, panitumumab, bevacizumab, rituximab, ibritumomab tiuxetan, gemtuzumab ozogamicin, denosumab, nivolumab, etc.; mTOR inhibitors such as temsirolimus, everolimus, rapamycin, etc.; proteasome inhibitors such as bortezomib, etc.; vitamin A derivatives such as tretinoin, tamibarotene, etc.; pyrimidine antagonists such as fluorouracil, tegafur-uracil, tegafur-gimeracil-oteracil potassium, capecitabine, cytarabine, gemcitabine, enocitabine, carmofur, etc.; purine antagonists such as mercaptopurine, fludarabine, cladribine, etc.; folic acid antagonists such as methotrexate, pemetrexed, etc.; metabolic antagonists such as trifluridine-tipiracil hydrochloride, nelarabine, pentostatin, etc.; platinum compounds such as cisplatin, carboplatin, oxaliplatin, nedaplatin, etc.; alkalizers such as cyclophosphamide, ifosfamide, melphalan, dacarbazine, temozolomide, nimustine, busulfan, etc.; antibiotics such as doxorubicin, epirubicin, amrubicin, idarubicin, daunorubicin, mitoxantrone, bleomycin, mitomycin C, actinomycin D, L-asparaginase, aclarubicin, pirarubicin, peplomycin, etc.; topoisomerase inhibitors such as irinotecan, nogitecan, etoposide, etc.; antimicrotubule agents such as vincristine, vindesine, vinblastine, vinorelbine, eribulin, paclitaxel, docetaxel, etc.; hormonal drugs such as tamoxifen, toremifene, anastrozole, letrozole, exemestane, goserelin, leuprorelin, ethynylestradiol, chlormadinone, bicalutamide, flutamide, prednisolone, etc., and these may be used alone or may be used in combination of two or more kinds.

In one embodiment of the present invention, the composition described in the present specification is a medium composition.

When the composition described in the present specification is used as a medium composition, which are not limited to these, there may further contain, for example, cell culture media; sodium, potassium, calcium, magnesium, phosphorus, chlorine, various kinds of amino acids, various kinds of vitamins, antibiotics, sera, fatty acids, sugars, bioactive compounds, other chemical components, other biological components, etc.

As the cell culture medium, for example, well-known materials, commercially available materials, etc., can be used. As the commercially available cell culture medium, which are not limited to these, there may be mentioned, for example, Dulbecco's Modified Eagle's medium (Dulbecco's Modified Eagle's Medium; DMEM), Ham's F12 medium (Ham's Nutrient Mixture F12), DMEM/F12 medium, McCoy's 5A medium (McCoy's 5A medium), Eagle's MEM (Eagle's Minimum Essential Medium; EMEM), αMEM (alpha Modified Eagle's Minimum Essential Medium; αMEM), MEM (Minimum Essential Medium), RPMI1640 medium, Iscove's Modified Dulbecco's medium (Iscove's Modified Dulbecco's Medium; IMDM), MCDB131 medium, William's medium E, IPL41 medium, Fischer's medium, StemPro34 (manufactured by Thermo Fisher Scientific K.K.), X-VIVO 10 (manufactured by Cambrex), X-VIVO 15 (manufactured by Cambrex), HPGM (manufactured by Cambrex), StemSpan H3000 (manufactured by STEMCELL Technologies, Inc.), StemSpanSFEM (manufactured by STEMCELL Technologies, Inc.), Stemlinell (manufactured by Sigma-Aldrich Co. LLC), QBSF-60 (manufactured by Quality Biological Inc.), StemProhESCSFM (manufactured by Thermo Fisher Scientific K.K.), Essential8 (Registered Trademark) medium (manufactured by Gibco), mTeSR1 or 2 medium (manufactured by STEMCELL Technologies, Inc.), TeSR-E8 medium (manufactured by STEMCELL Technologies, Inc.), Repro FF or Repro FF2 (manufactured by ReproCELL), Primate ES Cell Medium (manufactured by ReproCELL), PSGro hESC/iPSC medium (manufactured by System Biosciences LLC), Nutri Stem (Registered Trademark) medium (manufactured by Biological Industries), StemFit (Registered Trademark) medium (manufactured by AJINOMOTO CO., INC.), CSTI-7 medium (manufactured by Cell Science & Technology Institute, Inc.), MesenPRO RS medium (manufactured by Gibco), MF-Medium (Registered Trademark) mesenchymal stem cell-growing medium (manufactured by TOYOBO CO., LTD.), medium for mesenchymal stem cell (manufactured by PromoCell GmbH), medium 2 for mesenchymal stem cell (manufactured by PromoCell GmbH), MSCGM (manufactured by Lonza), MesenCult XF (manufactured by STEMCELL Technologies, Inc.), StemPro MSC XF (manufactured by Thermo Fisher Scientific K.K.), Sf-900II (manufactured by Thermo Fisher Scientific K.K.), Opti-Pro (manufactured by Thermo Fisher Scientific K.K.), etc.

A kind of additives such as various kinds of amino acids, various kinds of vitamins, antibiotics, sera, fatty acids, sugars, bioactive compounds, other chemical components, other biological components, etc., can be appropriately selected by those skilled in the art depending on the purpose.

An amount of cell culture medium or additives to be used can be appropriately selected by those skilled in the art depending on the purpose.

When the composition described in the present specification is used as a medium composition, for example, it may be in the form of a powder state, lyophilized product, liquid state, etc., by a method well-known to those skilled in the art together with the above-mentioned additives, etc.

A content of the compound having the structure of the formula I, or a metal complex thereof, or a pharmaceutically acceptable salt thereof, or the compound having the structure of the formula II, or a pharmaceutically acceptable salt thereof in the composition described in the present specification (for example, a medium composition) or a material in which the above is made in the form of a powder state, etc., can be appropriately controlled for those skilled in the art in consideration of, for example, the final concentration of the compound in the cell culture medium.

When the composition described in the present specification is used as a medium composition, for example, it may be applied to cell culture by previously mixing the cell culture medium, etc., with the composition described in the present specification (or compound), or else, the composition described in the present specification (or compound) may be added to the place where the cells are cultured in the cell culture medium.

The composition described in the present specification (or compound) may suppress secretion of extracellular vesicles from cells, tissues, etc., by bringing it into contact with the cells, tissues, etc., in vitro, ex vivo, etc. Accordingly, in another embodiment of the present invention, it is provided a method for suppressing secretion of extracellular vesicles, which comprises bringing the composition described in the present specification (or compound) into contact with cells or tissues in vitro or ex vivo.

### EXAMPLES

Hereinafter, the present invention will be explained in more detail by referring to Examples, but these Examples do not limit the scope of the present invention at all.

### Example 1

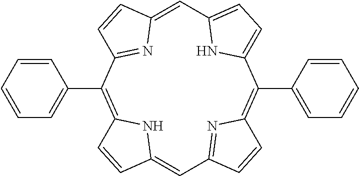 [SMILES: C1=Cc2nc1cc1ccc([nH]1)c(-c1ccccc1)c1nc(cc3ccc([nH]3)c2-c2ccccc2)C=C1]

As the compound of Example 1, a compound produced by Sigma-Aldrich (Product code: D4071-10MG, CAS registered No.: 22112-89-6) was used.

### Example 2

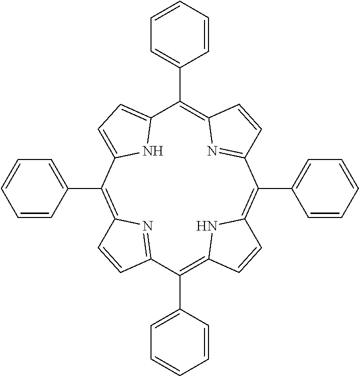 [SMILES: C1=Cc2nc1c(-c1ccccc1)c1ccc([nH]1)c(-c1ccccc1)c1nc(c(-c3ccccc3)c3ccc([nH]3)c2-c2ccccc2)C=C1]

As the compound of Example 2, a compound produced by Tokyo Chemical Industry Co., Ltd. (Product code: A5012, CAS registered No.: 917-23-7) was used.

### Example 3

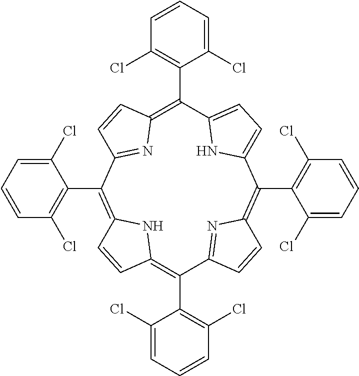 [SMILES: Clc1cccc(Cl)c1-c1c2nc(c(-c3c(Cl)cccc3Cl)c3ccc([nH]3)c(-c3c(Cl)cccc3Cl)c3nc(c(-c4c(Cl)cccc4Cl)c4ccc1[nH]4)C=C3)C=C2]

As the compound of Example 3, a compound produced by Tokyo Chemical Industry Co., Ltd. (Product code: T1438, CAS registered No.: 37083-37-7) was used.

### Example 4

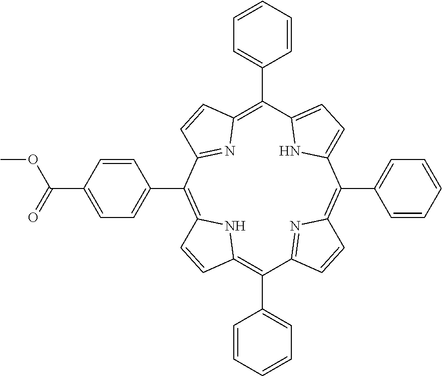 [SMILES: COC(=O)c1ccc(-c2c3nc(c(-c4ccccc4)c4ccc([nH]4)c(-c4ccccc4)c4nc(c(-c5ccccc5)c5ccc2[nH]5)C=C4)C=C3)cc1]

As the compound of Example 4, a compound produced by Tokyo Chemical Industry Co., Ltd. (Product code: M1338, CAS registered No.: 119730-06-2) was used.

### Example 5

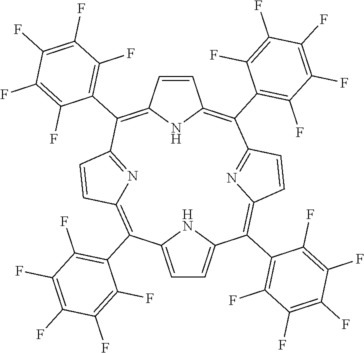 [SMILES: Fc1c(F)c(F)c(-c2c3nc(c(-c4c(F)c(F)c(F)c(F)c4F)c4ccc([nH]4)c(-c4c(F)c(F)c(F)c(F)c4F)c4nc(c(-c5c(F)c(F)c(F)c(F)c5F)c5ccc2[nH]5)C=C4)C=C3)c(F)c1F]

As the compound of Example 5, a compound produced by Tokyo Chemical

Industry Co., Ltd. (Product code: T1730, CAS registered No.: 25440-14-6) was used.

### Example 6

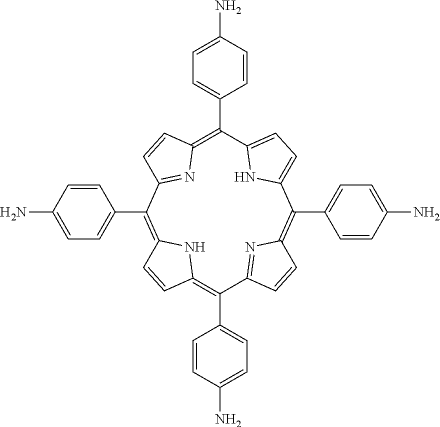 [SMILES: Nc1ccc(-c2c3nc(c(-c4ccc(N)cc4)c4ccc([nH]4)c(-c4ccc(N)cc4)c4nc(c(-c5ccc(N)cc5)c5ccc2[nH]5)C=C4)C=C3)cc1]

As the compound of Example 6, a compound produced by Tokyo Chemical Industry Co., Ltd. (Product code: T1494, CAS registered No.: 22112-84-1) was used.

### Example 7

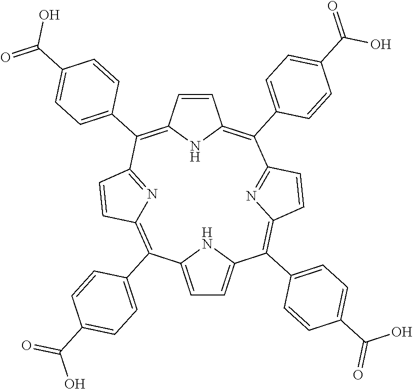 [SMILES: O=C(O)c1ccc(-c2c3nc(c(-c4ccc(C(=O)O)cc4)c4ccc([nH]4)c(-c4ccc(C(=O)O)cc4)c4nc(c(-c5ccc(C(=O)O)cc5)c5ccc2[nH]5)C=C4)C=C3)cc1]

As the compound of Example 7, a compound produced by Tokyo Chemical Industry Co., Ltd. (Product code: A5015, CAS registered No.: 14609-54-2) was used.

### Example 8

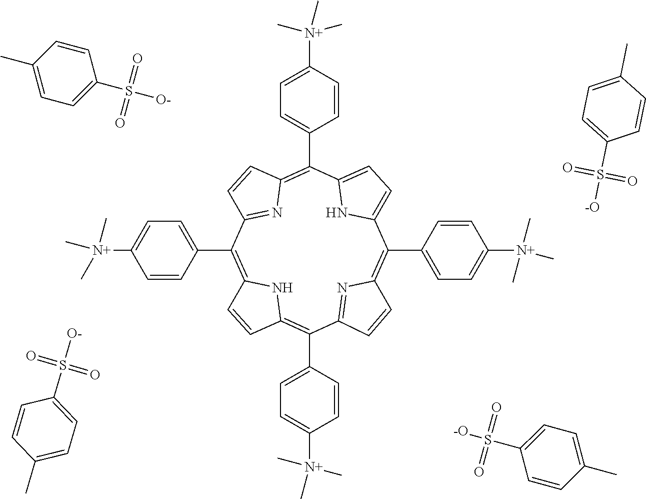

As the compound of Example 8, a compound produced by Kanto Chemical Co., Inc. (Product code: 31881-1A, CAS registered No.: 69458-20-4) was used.

### Comparative Example 1

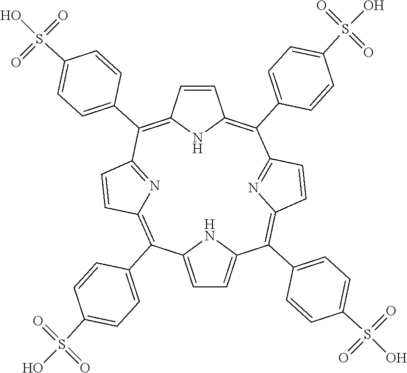 [SMILES: O=S(=O)(O)c1ccc(-c2c3nc(c(-c4ccc(S(=O)(=O)O)cc4)c4ccc([nH]4)c(-c4ccc(S(=O)(=O)O)cc4)c4nc(c(-c5ccc(S(=O)(=O)O)cc5)c5ccc2[nH]5)C=C4)C=C3)cc1]

As the compound of Comparative Example 1, a compound produced by Tokyo Chemical Industry Co., Ltd. (Product code: A5013, CAS registered No.: 35218-75-8) was used.

### Comparative Example 2

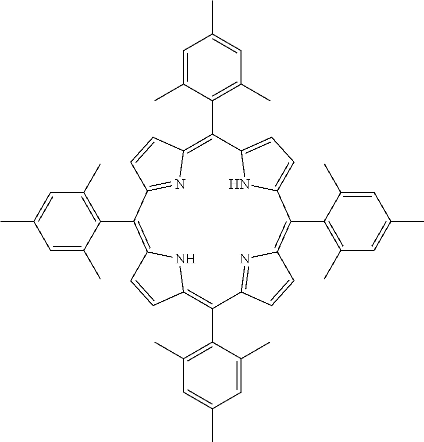

As the compound of Comparative Example 2, a compound produced by Tokyo Chemical Industry Co., Ltd. (Product code: T1729, CAS registered No.: 56396-12-4) was used.

### Comparative Example 3

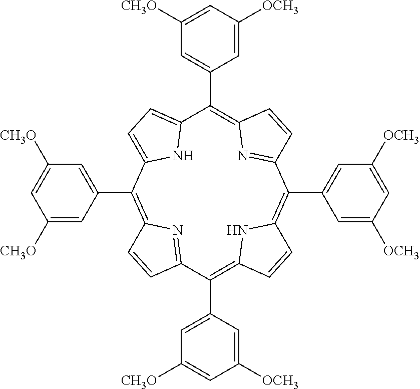

As the compound of Comparative Example 3, a compound produced by Tokyo Chemical Industry Co., Ltd. (Product code: T1832, CAS registered No.: 74684-34-7) was used.

### Comparative Example 4

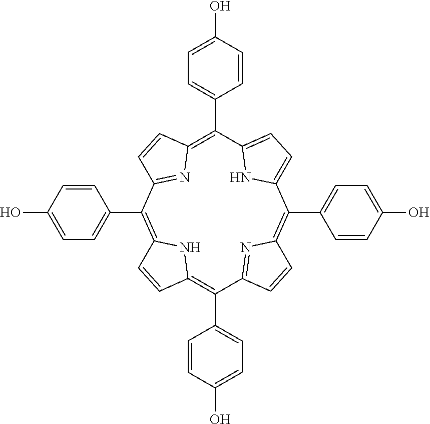 [SMILES: Oc1ccc(-c2c3nc(c(-c4ccc(O)cc4)c4ccc([nH]4)c(-c4ccc(O)cc4)c4nc(c(-c5ccc(O)cc5)c5ccc2[nH]5)C=C4)C=C3)cc1]

As the compound of Comparative Example 4, a compound produced by Tokyo Chemical Industry Co., Ltd. (Product code: T1497, CAS registered No.: 51094-17-8) was used.

### Comparative Example 5

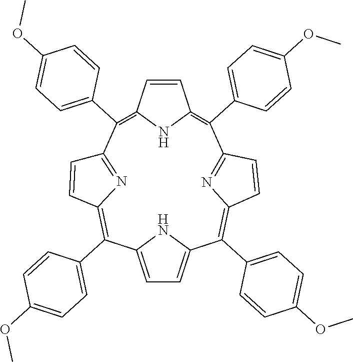 [SMILES: COc1ccc(-c2c3nc(c(-c4ccc(OC)cc4)c4ccc([nH]4)c(-c4ccc(OC)cc4)c4nc(c(-c5ccc(OC)cc5)c5ccc2[nH]5)C=C4)C=C3)cc1]

As the compound of Comparative Example 5, a compound produced by Tokyo Chemical Industry Co., Ltd. (Product code: T1360, CAS registered No.: 22112-78-3) was used.

### Example 9

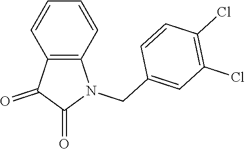 [SMILES: O=C1C(=O)N(Cc2ccc(Cl)c(Cl)c2)c2ccccc21]

As the compound of the compound of Example 9, a compound produced by MedChemExpress Co., Ltd. (Product code: HY-18633, CAS registered No.: 79183-19-0) was used.

### Example 10

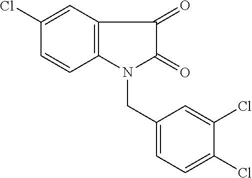 [SMILES: O=C1C(=O)N(Cc2ccc(Cl)c(Cl)c2)c2ccc(Cl)cc21]

As the compound of Example 10, a compound produced by BIONET (Product code: 12F-321S, CAS registered No.: 79183-32-7) was used.

### Example 11

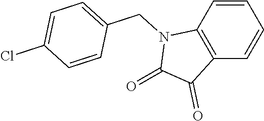 [SMILES: O=C1C(=O)N(Cc2ccc(Cl)cc2)c2ccccc21]

As the compound of Example 11, a compound produced by Enamine Ltd. (Product code: EN300-01090, CAS registered No.: 26960-66-7) was used.

### Example 12

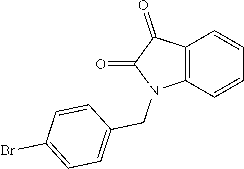 [SMILES: O=C1C(=O)N(Cc2ccc(Br)cc2)c2ccccc21]

As the compound of Example 12, a compound produced by Enamine Ltd. (Product code: Z57823200, CAS registered No.: 79183-37-2) was used.

### Example 13

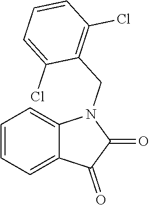 [SMILES: O=C1C(=O)N(Cc2c(Cl)cccc2Cl)c2ccccc21]

As the compound of Example 13, a compound produced by Pharmeks Ltd. (Product code: P2001S-151323, CAS registered No.: 303997-00-4) was used.

### Example 14

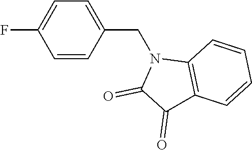 [SMILES: O=C1C(=O)N(Cc2ccc(F)cc2)c2ccccc21]

As the compound of Example 14, a compound produced by Pharmeks Ltd. (Product code: P2000S-19771, CAS registered No.: 313245-18-0) was used.

### Example 15

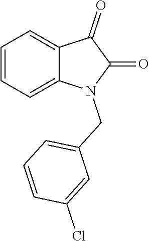 [SMILES: O=C1C(=O)N(Cc2cccc(Cl)c2)c2ccccc21]

As the compound of Example 15, a compound produced by Vitas-M (Product code: STL163705, CAS registered No.: 206537-57-7) was used.

### Example 16

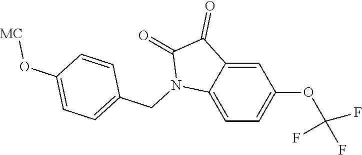

As the compound of Example 16, a compound produced by MedChemExpress Co., Ltd. (Product code: HY-12157, CAS registered No.: 1160247-92-6) was used.

### Comparative Example 6

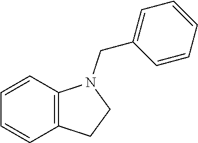 [SMILES: c1ccc(CN2CCc3ccccc32)cc1]

As the compound of Comparative Example 6, a compound produced by Enamine Ltd. (Product code: Z54749712, CAS registered No.: 6037-73-6) was used.

### Comparative Example 7

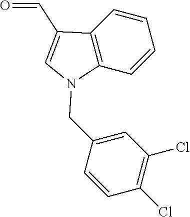 [SMILES: O=Cc1cn(Cc2ccc(Cl)c(Cl)c2)c2ccccc12]

As the compound of Comparative Example 7, a compound produced by Vitas-M (Product code: STK073558, CAS registered No.: 90815-02-4) was used.

### Comparative Example 8

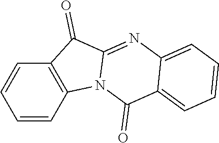 [SMILES: O=C1c2ccccc2-n2c1nc1ccccc1c2=O]

As the compound of Comparative Example 8, a compound produced by Pharmeks Ltd. (Product code: P2000N-00239, CAS registered No.: 13220-57-0) was used.

### Test Example 1-1: Evaluation 1 of Suppression of Secretion of Extracellular Vesicles

### <Preparation of Culture Supernatant>

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, U-87MG cells (HTB-14TM available from ATCC) were suspended in Advanced DMEM (#12491015 available from Thermo Fisher Scientific K.K.) containing 0.03% SphereMax (trademark) (available from Nissan Chemical Corporation) and 2% exosome-free FBS with 2×10⁴ cells/81 μL, and seeded on an ultra-low adhesive surface 384-well black clear bottom plate (#3827 manufactured by Corning Inc.). To the seeded U-87 MG cells was added 5 to 10 μL (final concentration: 0 to 10 μM; 0 μM corresponds to “solvent alone”) of each of the compounds of Examples 1 to 16 or Comparative Examples 1 to 8 prepared in Advanced DMEM (containing 2% exosome-free FBS), and the cells were cultured for 24 hours. Thereafter, the plate was centrifuged under the conditions of 1,200×g at 4° C. for 1 hour to precipitate the cells, and the culture supernatant was recovered. For the detection of extracellular vesicles contained in the culture supernatant, the Tim4-CD63 ELISA method or Tim4-CD9 ELISA method mentioned later was used.

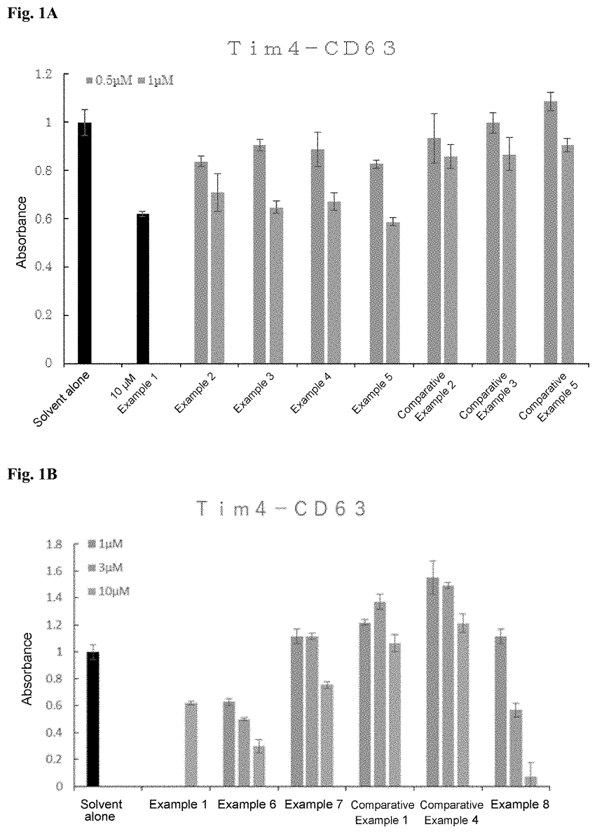

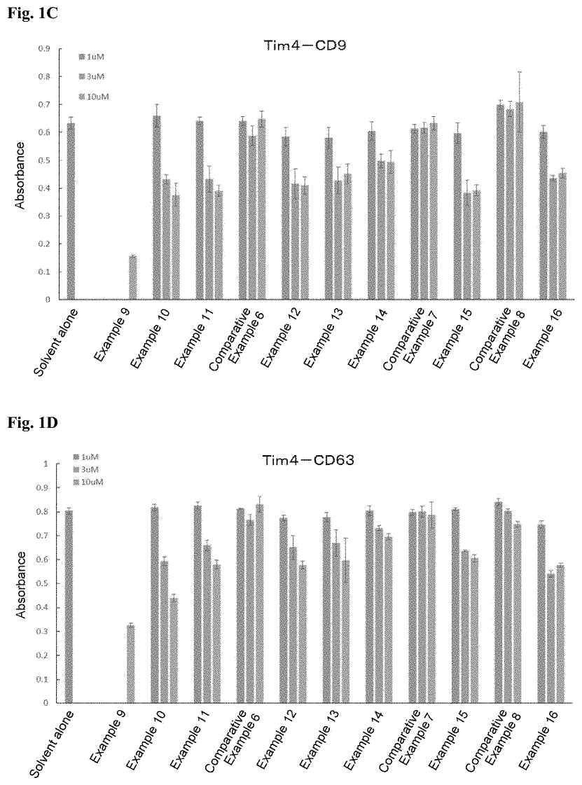

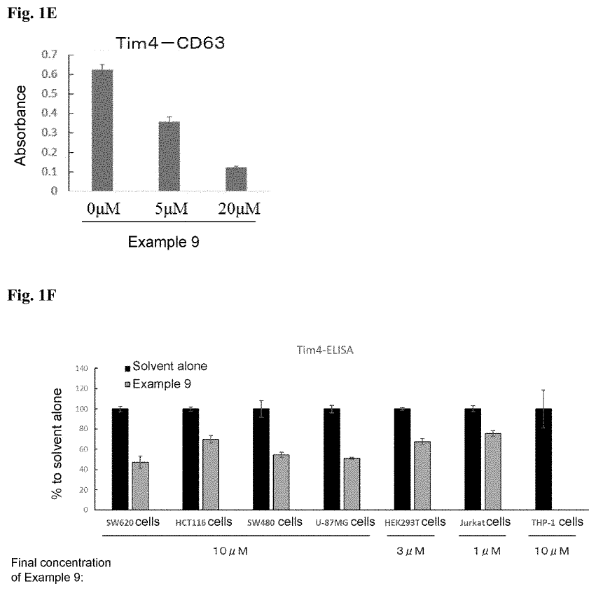

The results are shown in FIG. 1A to FIG. 1D.

### <Tim4-CD63 ELISA Method>

To a 384-well plate (#464718 manufactured by Thermo Fisher Scientific K.K.) was added 1 μg/mL Tim4 protein prepared in Carbonate Buffer (a solution containing 71.4 mM NaHCO₃ and 28.6 mM Na₂CO₃), and it was solidified. The 384-well plate was washed with a 0.05% Tween 20-containing TBS (Tris-Buffer Saline) solution (hereinafter sometimes referred to as “TBST solution”.), and then, 50 μL/well of a TBST solution containing 1% BSA was added thereto to carry out a blocking treatment for 1 hour. Next, 30 μL of the culture supernatant recovered above was added to the well, and further 10 μL of an 8 mM CaCl₂ solution was added thereto to bind extracellular vesicles to the Tim4 protein. Subsequently, 30 μL/well of 1 μg/mL Anti-human CD63 Antibody (#353014 available from BioLegend Inc.) diluted with a TBST solution containing 2 mM CaCl₂ was added thereto and the mixture was reacted for 1 hour. To the mixture was added 50 μL/well of a TBST solution containing 2 mM CaCl₂, after which the TBST solution was removed. After repeating this operation three times, 30 μL/well of 80 ng/mL HRP-conjugated Anti-mouse IgG (#405306 available from BioLegend Inc.) diluted with a TBST solution containing 2 mM CaCl₂) was added thereto and the mixture was reacted for 1 hour. Finally, 30 μL/well of a TMB solution (#05298-80 available from Nacalai Tesque Inc.) was added to the mixture and the mixture was reacted for 30 minutes, and then, 30 μL/well of a 1M H₂SO₄ solution was added thereto to stop the reaction and by measuring an absorbance at 450 nm to detect extracellular vesicles.

### <Tim4-CD9 ELISA Method>

The Tim4-CD9 ELISA method was carried out in the same manner except that 1 μg/mL Anti-human CD63 Antibody in the above-mentioned Tim4-CD63 ELISA method was replaced with 0.5 μg/mL Anti-human CD9 Antibody (#312102 available from BioLegend Inc.).

### Test Example 1-2: Evaluation 2 of Suppression of Secretion of Extracellular Vesicles

### <Preparation of Supernatant>

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, U-87 MG cells (HTB-14TM available from ATCC) were suspended in Advanced DMEM (#12491015 available from Thermo Fisher Scientific K.K.) containing 0.03% SphereMax (trademark) (available from Nissan Chemical Corporation) and 2% exosome-free FBS with 3×10⁵ cells/998 μL, and seeded on a non-adhesive surface 12-well plate (#665102 manufactured by Greiner Bio-One). To the seeded U-87 MG cells was added 2.5 or 10 mM of the compound of Example 9 prepared in 2 μL of DMSO, and the cells were cultured for 24 hours. Thereafter, the culture medium was recovered in 1.5 mL of a tube, the cells were precipitated under the centrifugation conditions of 300×g at 4° C. for 5 minutes, and the culture supernatant was recovered. Subsequently, the recovered culture supernatant was centrifugated under the conditions of 2,000×g at 4° C. for 20 minutes to precipitate cell fragments, and the supernatant was recovered. Further, the recovered supernatant was centrifuged under the conditions of 10,000×g at 4° C. for 30 minutes, and the supernatant was recovered. At this time, as a comparative subject, a compound-untreated group (0 μM, solvent alone) was also treated in the same manner as mentioned above, and the supernatant was recovered. For detection of extracellular vesicles contained in the supernatant after centrifugation of 10,000×g, the Tim4-CD63 ELISA method mentioned later was used.

The results are shown in FIG. 1E.

### <Tim4-CD63ELISA Method>

To a 96-well plate (#3801-096 manufactured by IWAKI & Co., Ltd.) was added 100 μL/well of 1 μg/mL Tim4 protein prepared in Carbonate Buffer (a solution containing 71.4 mM NaHCO₃ and 28.6 mM Na₂CO₃), and it was solidified. After washing the 96-well plate with a TBST solution, 200 μL/well of a TBST solution containing 1% BSA was added thereto to carry out a blocking treatment for 1 hour. Next, 90 μL of the supernatant recovered above was added to the well, and 10 μL of a 20 mM CaCl₂ solution was further added thereto to bind extracellular vesicles to the Tim4 protein. Subsequently, 100 μL/well of 1 μg/mL Anti-human CD63 Antibody (#353014 available from BioLegend Inc.) diluted with a TBST solution containing 2 mM CaCl₂ was added thereto and the mixture was reacted for 1 hour. To the mixture was added 200 μL/well of a TBST solution containing 2 mM CaCl₂, after which the TBST solution was removed. After repeating this operation three times, 100 μL/well of 80 ng/mL HRP-conjugated Anti-mouse IgG (#405306 available from BioLegend Inc.) diluted with a TBST solution containing 2 mM CaCl₂) was added thereto and the mixture was reacted for 1 hour. Finally, 100 μL/well of a TMB solution (#05298-80 available from Nacalai Tesque, Inc.) was added to the mixture and the mixture was reacted for 30 minutes, and then, 100 μL/well of a 1M H₂SO₄ solution was added thereto to stop the reaction and by measuring an absorbance at 450 nm to detect extracellular vesicles.

### Test Example 1-3: Evaluation of Suppression of Extracellular Vesicle Secretion in Various Cells

### <Preparation of Supernatant>

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, SW620 cells (ATCC, CCL-227), HCT116 cells (ATCC, CCL-247), SW480 cells (ATCC, CCL-228), U-87 MG cells (ATCC, HTB-14TM) or HEK293T cells (ATCC, CRL-3216) were suspended in Advanced DMEM (#12491015 available from Thermo Fisher Scientific K.K.) containing 2% exosome-free FBS with 2×10⁴ to 6×10⁴ cells/200 μL, and seeded on an adhesive surface 96-well plate (#3595 manufactured by Corning Inc.) and preculture for 24 hours was carried out. After 24 hours, the culture medium was removed, and a mixed solution of 199.6 μL of Advanced DMEM containing 2% exosome-free FBS and 0.4 μL of 0.5 to 5 mM of the compound of Example 9 prepared in DMSO was added, and the cells were cultured for 24 hours. Thereafter, the culture medium was recovered in 1.5 mL of a tube, the cells were precipitated under the centrifugation conditions of 300×g at 4° C. for 5 minutes, and the supernatant was recovered. Subsequently, the recovered supernatant was centrifuged under the centrifugation conditions of 2,000×g at 4° C. for 20 minutes to precipitate cell fragments, and the supernatant was recovered. Further, the recovered supernatant was centrifuged under the conditions of 10,000×g at 4° C. for 30 minutes, and the supernatant was recovered. At this time, as a comparative subject, a compound-untreated group (solvent alone, 0 μM) was also treated in the same manner as mentioned above, and the supernatant was recovered.

Recovery of the culture supernatant from Jurkat cells (RIKEN, RCB0806) or THP-1 cells (ATCC, TIB-202) was carried out in the same manner except that Advanced DMEM in the above-mentioned method was replaced with Advanced RPMI1640 (#12633012 available from Thermo Fisher Scientific K.K.).

For the detection of extracellular vesicles contained in the supernatant after the above-mentioned centrifugation of 10,000×g, the TIm4-ELISA method (Tim4-human CD9 ELISA method, Tim4-human CD63 ELISA method, Tim4-human CD81 ELISA method, Tim4-mouse CD63 ELISA method, or Tim4-mouse CD81 ELISA method) mentioned later was used.

The results are shown in FIG. 1F.

### <Tim4-ELISA Method>

To a 96-well plate (#3801-096 manufactured by IWAKI & Co., Ltd.) was added 100 μL/well of 1 μg/mL Tim4 protein prepared in Carbonate Buffer (a solution containing 71.4 mM NaHCO₃ and 28.6 mM Na₂CO₃), and it was solidified. After the 96-well plate was washed with a TBST solution, 200 μL/well of a TBST solution containing 1% BSA was added thereto to carry out a blocking treatment for 1 hour. Next, 90 μL of the supernatant recovered as mentioned above was added to the well, and 10 μL of 20 mM CaCl₂ solution was further added thereto to bind extracellular vesicles to the Tim4 protein. Subsequently, 100 μL/well of Primary antibody diluted with a TBST solution containing 2 mM CaCl₂ was added thereto to react the mixture for 1 hour. To the mixture was added 200 pt/well of a TBST solution containing 2 mM CaCl₂, after which the TBST solution was removed. After repeating this operation three times, 100 μL/well of Secondary antibody diluted with a TBST solution containing 2 mM CaCl₂ was added thereto to react the mixture for 1 hour. Finally, 100 μL/well of a TMB solution (#05298-80 available from Nacalai Tesque, Inc.) was added to the mixture and the mixture was reacted for 30 minutes, and then, 100 μL/well of a 1M H₂SO₄ solution was added thereto to stop the reaction and by measuring an absorbance at 450 nm to detect extracellular vesicles. The measured value of the compound-untreated group was set to 100%, and extracellular vesicles when the compound of Example 9 was added were evaluated as % of the compound-untreated group. Incidentally, the combination of Primary antibody and Secondary antibody used for detecting extracellular vesicles in the culture supernatant of each cell are as follows.

Culture Supernatant of SW620 Cells

Primary antibody: 1 μg/mL Anti-human CD9 Antibody (#312102 available from BioLegend Inc.)

Secondary antibody: 80 ng/mL HRP-conjugated Anti-mouse IgG (#405306 available from BioLegend Inc.)

Culture Supernatants of HCT116 Cells, SW480 Cells and U-87 MG Cells

Primary antibody: 1 μg/mL Anti-human CD63 Antibody (#353014 available from BioLegend Inc.)

Secondary antibody: 80 ng/mL HRP-conjugated Anti-mouse IgG

Culture Supernatants of HEK293T Cells, Jurkat Cells and THP-1 Cells

Primary antibody: 1 μg/mL Anti-human CD81Antibody (#349501 available from BioLegend Inc.)

Secondary antibody: 80 ng/mL HRP-conjugated Anti-mouse IgG

### Test Example 2-1: Evaluation of Cytotoxicity

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, U-87 MG cells (HTB-14TM available from ATCC) were suspended in Advanced DMEM (#12491015 available from Thermo Fisher Scientific K.K.) containing 0.03% SphereMax (trademark) (available from Nissan Chemical Corporation) and 2% exosome-free FBS with 2×10⁴ cells/81 μL, and seeded on an ultra-low adhesive surface 384-well black clear bottom plate (#3827 manufactured by Corning Inc.). To the seeded U-87 MG cells were added 5 to 10 μL (final concentration: 0 to 40 μM) of each of the compounds of Examples 1 to 16 or Comparative Examples 1 to 8 prepared in Advanced DMEM (containing 2% exosome-free FBS), and the cells were cultured for 24 hours. Thereafter, the plate was centrifuged under the conditions of 1200×g at 4° C. for 1 hour to precipitate the cells, and 20 μL of the culture supernatant was transferred to a 384-well microplate (#781101 available from Greiner Bio-One). The activity of lactate dehydrogenase (LDH) contained in the culture supernatant was measured by using Cytotoxicity LDH Assay Kit-WST (#CK12 manufactured by Dojindo Laboratories). 5 mL of Assay Buffer was added to 500 tests of Dye Mixture and dissolved to prepare Working Solution. To the 384-well microplate to which 20 μL of the culture supernatant was transferred was added 20 μL of the Working Solution, and after mixing well, a coloring reaction was carried out at room temperature for 30 minutes. The reaction was stopped by adding 10 μL of Stop Solution, and the cytotoxicity was evaluated by measuring the absorbance at 490 nm. The activity of LDH was also measured in the dead cell group (sample to which 9 μL of Lysis Buffer was added to compound-untreated cells 15 minutes before recovery of the culture medium, PC). With regard to a part of the tests, the measured value of PC was evaluated as 100% cytotoxicity, and cytotoxicity by each compound was evaluated as % of absorbance relative to PC.

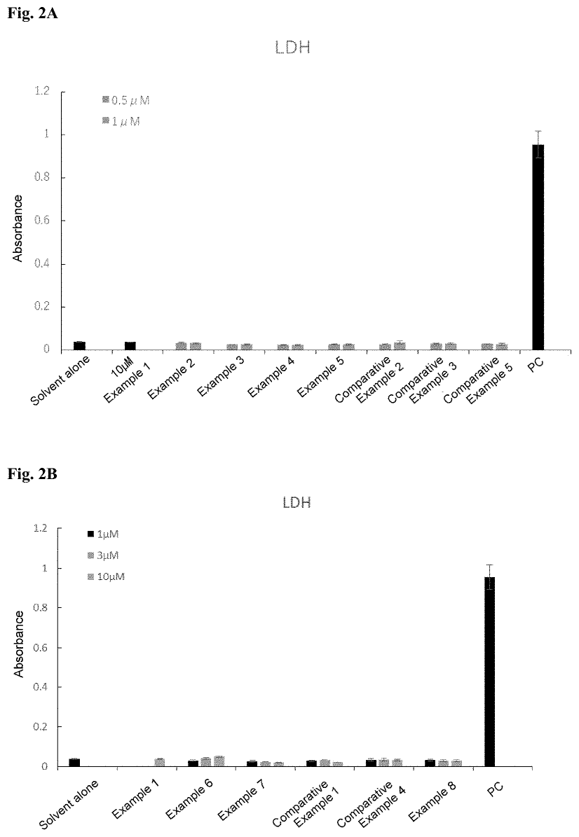

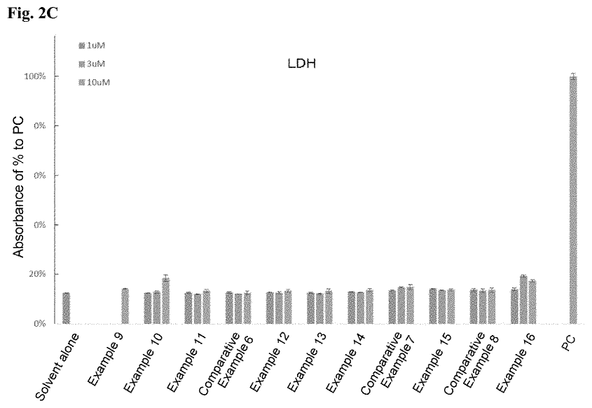

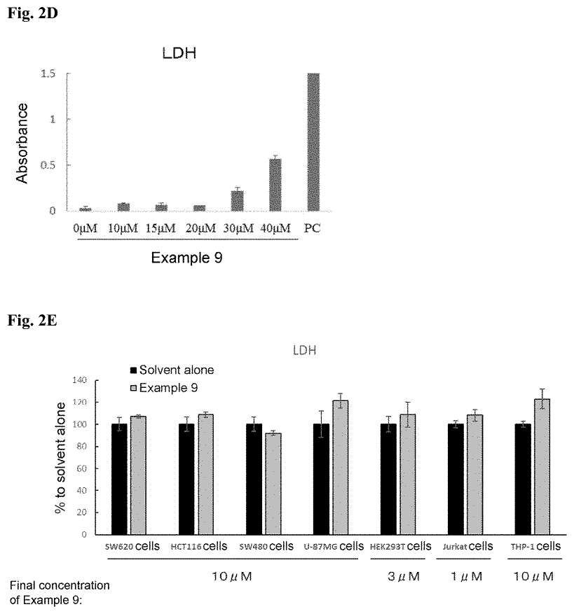

The results are shown in FIG. 2A to FIG. 2D.

### Test Example 2-2: Evaluation of Cytotoxicity in Various Cells

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, SW620 cells (ATCC, CCL-227), HCT116 cells (ATCC, CCL-247), SW480 cells (ATCC, CCL-228), U-87 MG cells (ATCC, HTB-14TM) or HEK293T cells (ATCC, CRL-3216) were suspended in Advanced DMEM (#12491015 available from Thermo Fisher Scientific K.K.) containing 2% exosome-free FBS with 2×10⁴ to 6×10⁴ cells/100 μL, and seeded on an adhesive surface 96-well plate (#3595 manufactured by Corning Inc.) and preculture for 24 hours was carried out. After 24 hours, the culture medium was removed, and a mixed solution of 199.6 μL of Advanced DMEM containing 2% exosome-free FBS and 0.4 μL of 0.5 to 5 mM of the compound of Example 9 prepared in DMSO was added, and the cells were cultured for 24 hours. At this time, as a comparative subject, a compound-untreated group (solvent alone, 0 μM) was also treated in the same manner as mentioned above, and culture was carried out. Thereafter, the plate was centrifuged under the conditions of 1200×g at 4° C. for 1 hour to precipitate the cells, and 50 μL of the culture supernatant was transferred to a 96-well microplate (#195-96F manufactured by WATSON).

Recovery of the culture supernatant from Jurkat cells (RIKEN, RCB0806) or THP-1 cells (ATCC, TIB-202) was carried out in the same manner except that Advanced DMEM in the above-mentioned method was replaced with Advanced RPMI1640 (#12633012 available from Thermo Fisher Scientific K.K.).

The activity of lactate dehydrogenase (LDH) contained in the culture supernatant was measured by using Cytotoxicity LDH Assay Kit-WST (#CK12 manufactured by Dojindo Laboratories). 5 mL of Assay Buffer was added to 500 tests of Dye Mixture and dissolved to prepare Working Solution. To the 96-well microplate to which 50 μL of the culture supernatant was transferred was added 50 μL of the Working Solution, and after mixing well, a coloring reaction was carried out at room temperature for 30 minutes. The reaction was stopped by adding 25 μL of Stop Solution, and the cytotoxicity was evaluated by measuring the absorbance 490 nm. The measured value of the compound-untreated group was made 100%, and cytotoxicity of the compound of Example 9 was evaluated as % of that of the compound-untreated group.

The results are shown in FIG. 2E.

### Test Example 3: Evaluation 3 of Suppression of Secretion of Extracellular Vesicles

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, U-87MG cells (HTB-14TM available from ATCC) were suspended in Advanced DMEM (#12491015 available from Thermo Fisher Scientific K.K.) containing 0.03% SphereMax (trademark) (available from Nissan Chemical Corporation) and 2% exosome-free FBS with 3×10⁵ cells/998 μL, and seeded to a non-adhesive surface 12-well plate (#665102 manufactured by Greiner Bio-One). To the seeded U-87 MG cells was added 2 μL (final concentration: 0 to 20 μM) of each of the compounds of Examples 9, 11 to 15 prepared in DMSO, and the cells were cultured for 24 hours. Thereafter, the culture medium was recovered in 1.5 mL of a tube, the cells were precipitated under the centrifugation conditions of 300×g at 4° C. for 5 minutes, and the culture supernatant was recovered. Subsequently, the recovered culture supernatant was centrifuged under the conditions of 2,000×g at 4° C. for 20 minutes to precipitate cell fragments, and the supernatant was recovered. Further, the recovered supernatant was centrifuged under the conditions of 10,000×g at 4° C. for 30 minutes, and the supernatant was recovered. At this time, as a comparative subject, the compound-untreated group (solvent alone, 0 μM) was also treated in the same manner as mentioned above, and the supernatant was recovered. An amount of extracellular vesicles contained in 500 μL of the supernatant after centrifugation of 10,000×g was quantitatively determined by using a nanoparticle analysis system (#NanoSIGHT LM10 manufactured by Malvern Panalytical).

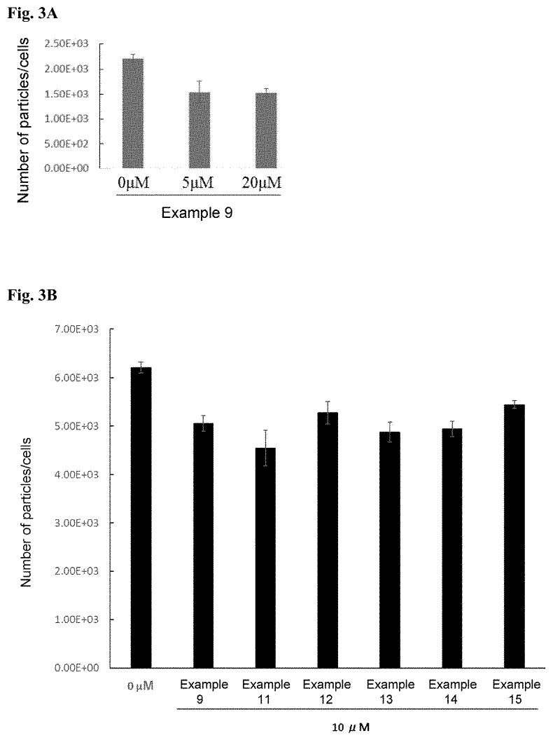

The results are shown in FIG. 3A and FIG. 3B.

### Test Example 4: Evaluation 4 of Suppression of Secretion of Extracellular Vesicles

### <Preparation of Supernatant>

After stirring 10 mL of deactivated FBS and 2 mL of a 50% Poly(ethylene glycol) 10,000 solution (#81280 available from Sigma-Aldrich Co., LLC) at 4° C. for 2 hours, extracellular vesicles were precipitated under the centrifugation conditions of 1,500×g at 4° C. for 30 minutes, and the supernatant was recovered to prepare exosome-free FBS. Next, U-87 MG cells (HTB-14TM available from ATCC) were suspended in Advanced DMEM (#12491015 available from Thermo Fisher Scientific K.K.) containing 2% exosome-free FBS with 3×10⁵ cells/1000 μL, and seeded to an adhesive surface 12-well plate (#3513 available from Corning Inc.), and preculture for 24 hours was carried out. After 24 hours, the culture medium was removed, a mixed solution of 998 μL of Advanced DMEM containing 2% exosome-free FBS and 2 μL of 2.5 or 10 mM of the compound of Example 9 prepared in DMSO was added thereto, and cultured for 2 hours. After 2 hours, the culture medium was removed, 1,000 μL of Advanced DMEM containing 2% exosome-free FBS was added thereto, and the cells were further cultured for 24 hours. Thereafter, the culture medium was recovered in 1.5 mL of a tube, the cells were precipitated under the centrifugation conditions of 300×g at 4° C. for 5 minutes, and the culture supernatant was recovered. Subsequently, the recovered culture supernatant was centrifugated under the conditions of 2,000×g at 4° C. for 20 minutes to precipitate cell fragments, and the supernatant was recovered. Further, the recovered supernatant was centrifuged under the conditions of 10,000×g at 4° C. for 30 minutes, and the supernatant was recovered. At this time, as a comparative subject, a compound-untreated group (solvent alone, 0 μM) was also treated in the same manner as mentioned above, and the supernatant was recovered. For the detection of extracellular vesicles contained in the supernatant after centrifugation of 10,000×g, the Tim4-CD63 ELISA method mentioned later was used.

### <Tim4-CD63ELISA Method>

To a 96-well plate (#3801-096 manufactured by IWAKI & Co., Ltd.) was added 100 μL/well of 1 μg/mL Tim4 protein prepared in Carbonate Buffer (a solution containing 71.4 mM NaHCO₃ and 28.6 mM Na₂CO₃), and it was solidified. After the 96-well plate was washed with a TBST solution, 200 μL/well of a TBST solution containing 1% BSA was added thereto to carry out a blocking treatment for 1 hour. Next, 90 μL of the supernatant recovered as mentioned above was added to the well, and 10 μL of 20 mM CaCl₂ solution was further added thereto to bind extracellular vesicles to the Tim4 protein. Subsequently, 100 μL/well of 1 μg/mL Anti-human CD63 Antibody (#353014 available from BioLegend Inc.) diluted with a TBST solution containing 2 mM CaCl₂ was added thereto to react the mixture for 1 hour. To the mixture was added 200 μL/well of a TBST solution containing 2 mM CaCl₂, after which the TBST solution was removed. After repeating this operation three times, 100 μL/well of 80 ng/mL HRP-conjugated Anti-mouse IgG (#405306 available from BioLegend Inc.) diluted with a TBST solution containing 2 mM CaCl₂) was added thereto to react the mixture for 1 hour. Finally, 100 μL/well of a TMB solution (#05298-80 available from Nacalai Tesque, Inc.) was added to the mixture and the mixture was reacted for 30 minutes, and then, 100 μL/well of a 1M H₂SO₄ solution was added thereto to stop the reaction and by measuring an absorbance at 450 nm to detect extracellular vesicles.

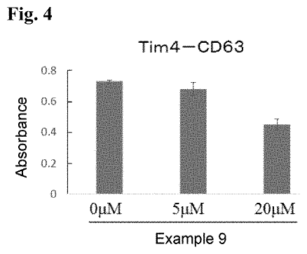

The results are shown in FIG. 4.

[Results]

From the results of Test Examples 1, 3 and 4, it can be understood that the compounds of Examples 1 to 16 could reduce the secreted amount of CD9, CD63 and/or CD81-positive extracellular vesicles from various cells (for example, U-87 MG cells, HEK293T cells, Jurkat cells, THP-1 cells, HCT116 cells, SW480 cells and SW620 cells).

Further, from the results of Test Example 2, the compounds of Examples 1 to 16 are considered to be low cytotoxicity to various cells (for example, U-87 MG cells, HEK293T cells, Jurkat cells, THP-1 cells, HCT116 cells, SW480 cells and SW620 cells) since the amount of LDH in the culture supernatant is small.

Accordingly, the compositions containing the compounds of Examples 1 to 16 suppress secretion of extracellular vesicles from various cells (for example, U-87 MG cells, HEK293T cells, Jurkat cells, THP-1 cells, HCT116 cells, SW480 cells and SW620 cells). Further, the compositions containing the compounds of Examples 1 to 16 suppress secretion of extracellular vesicles from various cells (for example, U-87 MG cells, HEK293T cells, Jurkat cells, THP-1 cells, HCT116 cells, SW480 cells and SW620 cells) irrespective of having low cytotoxicity to these cells.

As mentioned above, from the results of Test Examples 1, 3 and 4, it can be understood that the compounds of Examples 1 to 16 suppress secretion of extracellular vesicles (preferably cancer cells or extracellular vesicles derived from cancer) derived from various cells or tissues. Accordingly, the compound having the structure of the formula I, or a metal complex thereof, or a pharmaceutically acceptable salt thereof, or the compound having the structure of the formula II, or a pharmaceutically acceptable salt thereof is a material that suppresses secretion of extracellular vesicles (preferably cancer cells or extracellular vesicles derived from cancer) derived from various cells or tissues.

Further, from the results of Test Example 2, in the compounds of Examples 1 to 16, no significant increase in the amount of LDH in the culture supernatant could be admitted. Accordingly, the compound having the structure of the formula I, or a metal complex thereof, or a pharmaceutically acceptable salt thereof, or the compound having the structure of the formula II, or a pharmaceutically acceptable salt thereof is considered to be a material which is low cytotoxicity at a concentration of, for example, about 40 μM or less (preferably about 20 μM or less, more preferably about 10 μM or less) as a concentration of the compound, and is safe.

Further, the compound having the structure of the formula I, or a metal complex thereof, or a pharmaceutically acceptable salt thereof, or the compound having the structure of the formula II, or a pharmaceutically acceptable salt thereof can suppress secretion of preferably cancer cells or extracellular vesicles derived from cancer as mentioned above, so that it can be considered that they can treat or prevent from cancer, or can prevent from cancerous metastasis.

1. A composition for suppressing secretion of extracellular vesicles, which comprises a compound having a structure of the formula II: 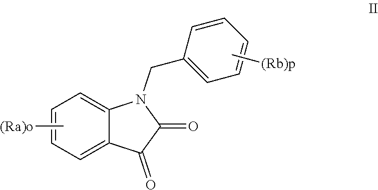
wherein
each Ra is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,
each Rb is independently halogen; hydroxy; amino; nitro; C₁₋₆ alkyl which may be substituted by halogen, hydroxy, amino or nitro; or C₁₋₆ alkoxy which may be substituted by halogen, hydroxy, amino or nitro,
o is 0 to 4, and
p is 0 to 5,
or a pharmaceutically acceptable salt thereof.

2. A composition for suppressing secretion of extracellular vesicles, which comprises a compound having a structure of the formula I: 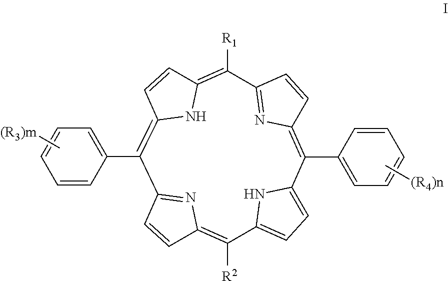
wherein
R₁ and R₂ are independently hydrogen, or phenyl which may be substituted by 1 to 5 substituents selected from the group consisting of halogen, —C(O)R₅, —NR₆R₇,
—N⁺R₈R₉R₁₀ and nitro,
each R₃ is independently halogen, —C(O)R₅, —NR₆R₇, —N⁺R₈R₉R₁₀ or nitro,
each R₄ is independently halogen, —C(O)R₅, —NR₆R₇, —N⁺R₈R₉R₁₀ or nitro,
each R₅ is independently hydrogen, hydroxy, or C₁₋₆ alkyl which may be substituted by halogen, hydroxy, amino or nitro,
each R₆, each R₇, each R₈, each R₉ and each R₁₀ are independently hydrogen, or C₁₋₆ alkyl which may be substituted by halogen, hydroxy, amino or nitro,
m is 0 to 5, and
n is 0 to 5,
or a metal complex thereof, or a pharmaceutically acceptable salt thereof.

3. The composition according to claim 1, wherein o is 0.

4. The composition according to claim, wherein p is 1 or 2.

5. The composition according to claim 1, wherein each Rb is halogen.

6. The composition according to claim 1, wherein the compound having the structure of the formula II is selected from the group consisting of the following:

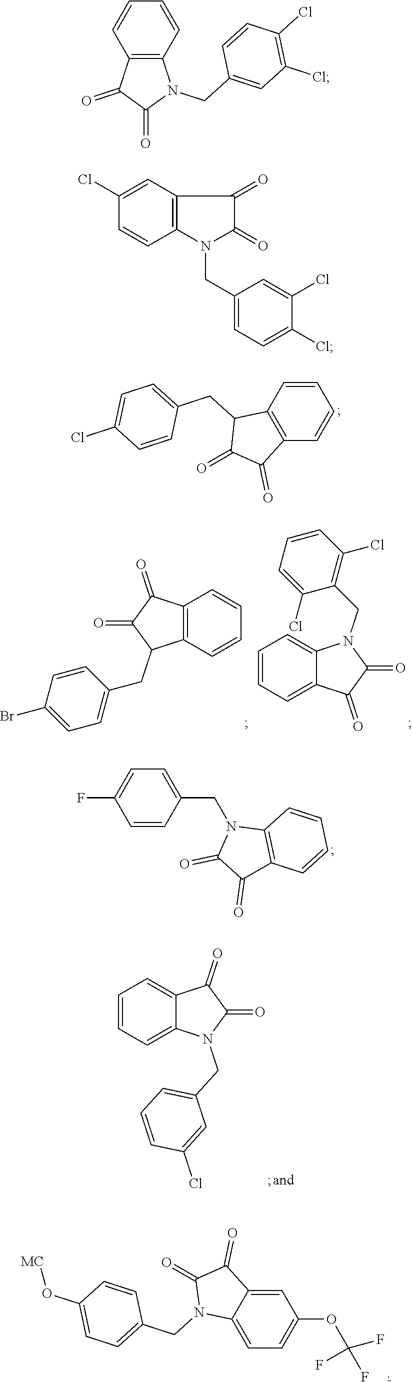

7. The composition according to claim 2, wherein R₁ and R₂ are phenyl which may be substituted by 1 to 5 substituents selected from the group consisting of halogen, —C(O)R₅, —NR₆R₇, —N⁺R₈R₉R₁₀ and nitro.

8. The composition according to claim 2, wherein m is 1.

9. The composition according to claim 2, wherein R₁ and R₂ are hydrogen.

10. The composition according to claim 2, wherein the compound having the structure of the formula I is selected from the group consisting of the following:

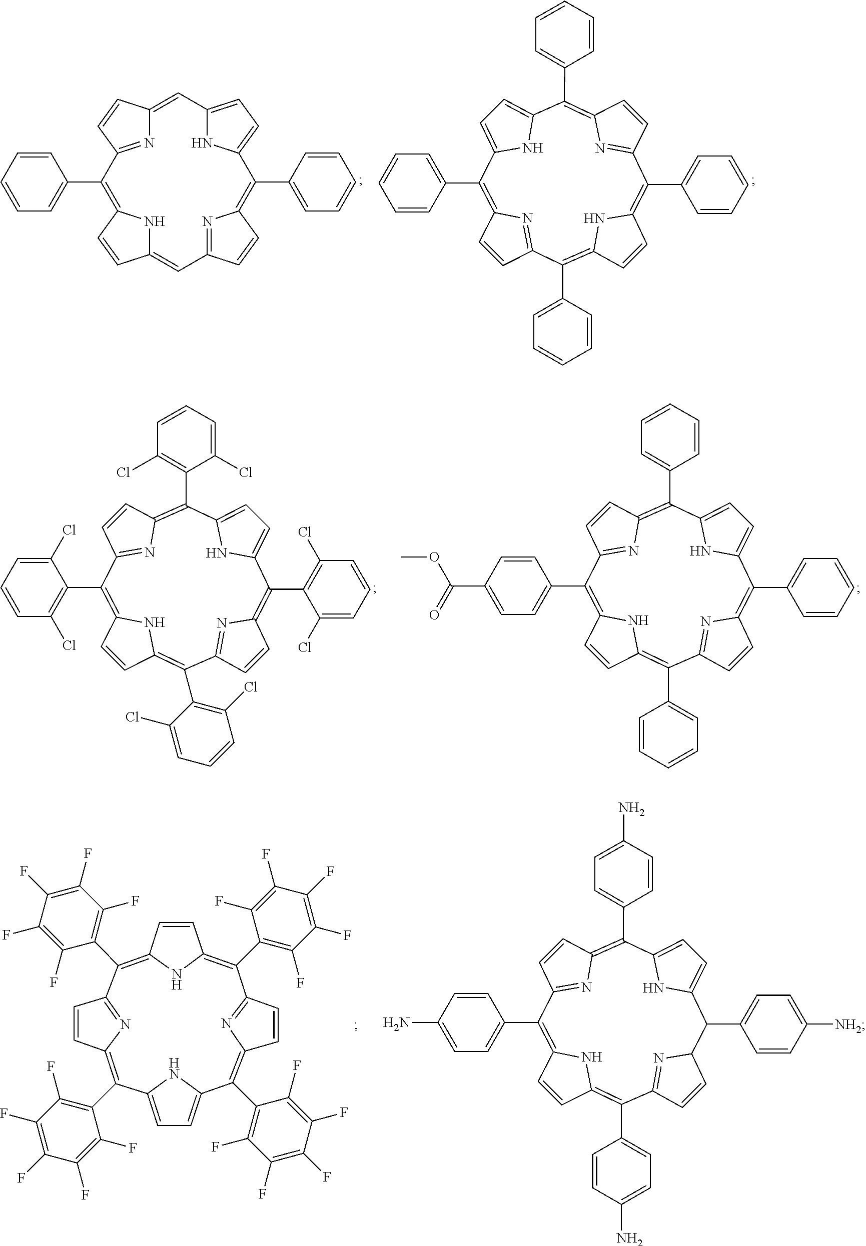

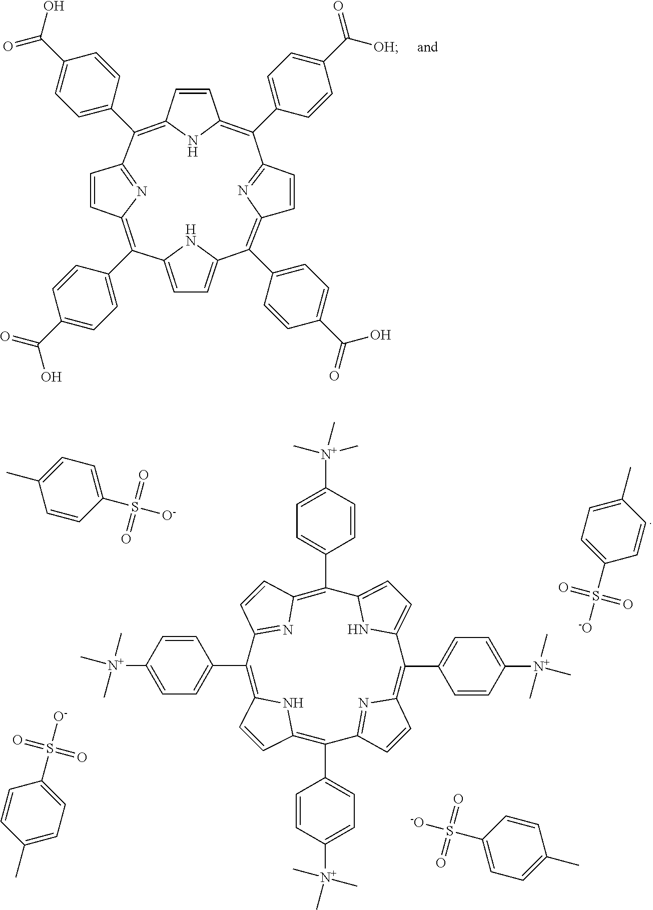

11. A method for treatment or prophylaxis of cancer comprising administering a composition according to claim 1 to a subject in need thereof.

12. A method for prophylaxis of cancerous metastasis comprising administering a composition according to claim 1 to a subject in need thereof.

13. The composition according to claim 1, which is a medium composition.

14. A method for suppressing secretion of extracellular vesicles, which comprises bringing the compound, or a metal complex thereof, or a pharmaceutically acceptable salt thereof defined in claim 1 into contact with cells or tissues in vitro or ex vivo.
